SEQUENCE LISTING (6 sequences)

SEQ ID NO: 1 - protein, 117 aa, Artificial Sequence
  Description of Artificial Sequence; note = synthetic construct
MPSPGTVCSLLLLGMLWLDLAMAGSSFLSPEHQRVQQRKESKKPPAKLQPRALAGWLRPEDGGQAEGAEDELEVRFNAPFDVGIKLSGVQYQQHSQALGKFLQDILWEEAKEAPADK

SEQ ID NO: 2 - DNA, 511 bp, Artificial Sequence
  Description of Artificial Sequence; note = synthetic construct
gcaggcccacctgtctgcaacccagctgaggccatgccctccccagggaccgtctgcagcctcctgctcctcggcatgctctggctggacttggccatggcaggctccagcttcctgagccctgaacaccagagagtccagcagagaaaggagtcgaagaagccaccagccaagctgcagccccgagctctagcaggctggctccgcccggaagatggaggtcaagcagaaggggcagaggatgaactggaagtccggttcaacgccccctttgatgttggaatcaagctgtcaggggttcagtaccagcagcacagccaggccctggggaagtttcttcaggacatcctctgggaagaggccaaagaggccccagccgacaagtgatcgcccacaagccttactcacctctctctaagtttagaagcgctcatctggcttttcgcttgcttctgcagcaactcccacgactgttgtacaagctcaggaggcgaataaatgttcaaactgt

SEQ ID NO: 3 - protein, 18 aa, Artificial Sequence
  Description of Artificial Sequence; note = synthetic construct
GSXFLSPEHQRVQQRKES

SEQ ID NO: 4 - protein, 14 aa, Artificial Sequence
  Description of Artificial Sequence; note = synthetic construct
GSXFLSPEHQRVQQ

SEQ ID NO: 5 - protein, 10 aa, Artificial Sequence
  Description of Artificial Sequence; note = synthetic construct
GSXFLSPEHQ

SEQ ID NO: 6 - protein, 5 aa, Artificial Sequence
  Description of Artificial Sequence; note = synthetic construct
GSXFL

(19) United States
(12) Patent Application Publication
(10) Pub. No.: US 2008/0269116 A1
(43) Pub. Date: Oct. 30, 2008

(54) Methods of Inhibiting Proinflammatory Cytokine Expression Using Ghrelin

(76) Inventors: Dennis D. Taub, Baltimore, MD (US); Vishwa Deep Dixit, Baton Rouge, LA (US)

Correspondence Address: NATIONAL INSTITUTE OF HEALTH;C/O Ballard Spahr Andrews & Ingersoll, LLP, SUITE 1000, 999 PEACHTREE STREET, ATLANTA, GA, 30309, US

(21) Appl. No.: 11/596,310

(22) PCT Filed: May 11, 2005
(86) PCT No.: PCT/US2005/016565

§ 371 Date: Jun. 6, 2008

Related U.S. Application Data
(60) Provisional application No. 60/569,819, filed on May 11, 2004.

(51) Int. Cl. A61K 38/00; A61P 3/00

(52) U.S. Cl. 514/12

(57) ABSTRACT

The present invention provides a method of inhibiting proinflammatory cytokine expression using ghrelin.

### BACKGROUND OF THE INVENTION

Inflammation is a complex stereotypical reaction of the body expressing the response to damage of cells and vascularized tissues. The discovery of the detailed processes of inflammation has revealed a close relationship between inflammation and the immune response. The main features of the inflammatory response are vasodilation, i.e. widening of the blood vessels to increase the blood flow to the infected area; increased vascular permeability, which allows diffusible components to enter the site; cellular infiltration by chemotaxis, or the directed movement of inflammatory cells through the walls of blood vessels into the site of injury; changes in biosynthetic, metabolic, and catabolic profiles of many organs; and activation of cells of the immune system as well as of complex enzymatic systems of blood plasma.

There are two forms of inflammation, acute and chronic. Acute inflammation can be divided into several phases. The earliest, gross event of an inflammatory response is temporary vasoconstriction, i.e. narrowing of blood vessels caused by contraction of smooth muscle in the vessel walls, which can be seen as blanching (whitening) of the skin. This is followed by several phases that occur minutes, hours and days later. The first is the acute vascular response, which follows within seconds of the tissue injury and lasts for several minutes. This results from vasodilation and increased capillary permeability due to alterations in the vascular endothelium, which leads to increased blood flow (hyperemia) that causes redness (erythema) and the entry of fluid into the tissues (edema).

The acute vascular response can be followed by an acute cellular response, which takes place over the next few hours. The hallmark of this phase is the appearance of granulocytes, particularly neutrophils, in the tissues. These cells first attach themselves to the endothelial cells within the blood vessels (margination) and then cross into the surrounding tissue (diapedesis). During this phase erythrocytes may also leak into the tissues and a hemorrhage can occur. If the vessel is damaged, fibrinogen and fibronectin are deposited at the site of injury, platelets aggregate and become activated, and the red cells stack together in what are called “rouleau” to help stop bleeding and aid clot formation. The dead and dying cells contribute to pus formation. If the damage is sufficiently severe, a chronic cellular response may follow over the next few days. A characteristic of this phase of inflammation is the appearance of a mononuclear cell infiltrate composed of macrophages and lymphocytes. The macrophages are involved in microbial killing, in clearing up cellular and tissue debris, and in remodeling of tissues.

Chronic inflammation is an inflammatory response of prolonged duration—weeks, months, or even indefinitely—whose extended time course is provoked by persistence of the causative stimulus to inflammation in the tissue. The inflammatory process inevitably causes tissue damage and is accompanied by simultaneous attempts at healing and repair. The exact nature, extent and time course of chronic inflammation is variable, and depends on a balance between the causative agent and the attempts of the body to remove it. Etiological agents producing chronic inflammation include: (i) infectious organisms that can avoid or resist host defenses and so persist in the tissue for a prolonged period, including Mycobacterium tuberculosis, Actinomycetes, and numerous fungi, protozoa and metazoal parasites. Such organisms are in general able to avoid phagocytosis or survive within phagocytic cells, and tend not to produce toxins causing acute tissue damage. (ii) Infectious organisms that are not innately resistant but persist in damaged regions where they are protected from host defenses. An example is bacteria which grow in the pus within an undrained abscess cavity, where they are protected both from host immunity and from blood-borne therapeutic agents, e.g. antibiotics. Some locations are particularly prone to chronic abscess formation, e.g. bone, and pleural cavities. (iii) Irritant non-living foreign material that cannot be removed by enzymatic breakdown or phagocytosis. Examples include a wide range of materials implanted into wounds (wood splinters, grit, metals and plastics), inhaled (silica dust and other particles or fibers), or deliberately introduced (surgical prostheses, sutures, etc.) Also included are transplants. Dead tissue components that cannot be broken down may have similar effects, e.g. keratin squames from a ruptured epidermoid cyst or fragments of dead bone (sequestrum) in osteomyelitis. (iv) In some cases the stimulus to chronic inflammation may be a normal tissue component. This occurs in inflammatory diseases where the disease process is initiated and maintained because of an abnormality in the regulation of the body's immune response to its own tissues—the so-called auto-immune diseases. This response is seen in elderly and aging subjects. (v) For many diseases characterized by a chronic inflammatory pathological process the underlying cause remains unknown. An example is Crohn's disease (FIG. 11).

Inflammation and activation of innate immunity are common responses to replication incompetent adenoviruses (Ad) which are used as vectors for gene therapy (Jooss, K. Gene Ther. 10:955-963 (2003); Zaiss, A. K. J. Virol. 76:4580-4590, (2002)). The complement system is central to both innate immunity and inflammation (Walport, M. J. N Eng J Med 344:1058-1066 and 1140-1144 (2001)). Because it is comprised of multiple membrane-bound and blood factors, the complement system is of particular relevance in delivery of vectors administered intravenously. In fact, Cichon et al. (Gene Ther 8:1794-1800 (2001)) showed complement was activated in a majority of human plasma samples when challenged with different adenoviral serotypes; complement activation was completely dependent on anti-Ad antibody (Cichon (2001)).

The complement mediated inactivation is a multistep enzymatic cascade which finally results in formation of a membrane attack complex (MAC) mediating the perforation of membranes and subsequent lysis of the invading organism. It is either initiated by antigen-antibody complexes (classical pathway) or via an antibody independent pathway which is activated by certain particular polysaccharides, viruses and bacteria (alternative pathway).

Human organs and cells themselves are protected to complement mediated lysis. This protection is achieved by expression of complement inactivation factors. So far, five human factors are known. CD35 (CR1) is released from the cells and acts mainly extrinsically. In contrast, CD59, CD46 (MCP), CD55 (DAF) and HRF are integrated into the cellular membrane. CD46 (MCP) is a classical transmembrane protein while HRF, CD59 and CD55 are GPI-anchored. These factors can interrupt the complement cascade at two different stages: DAF, CR1 and MCP act at an early stage of both the alternative and the classical pathway. In contrast, CD59 and HRF inhibit the assembly of the membrane attack complex, which is the final step of both pathways resulting in channel formation and lysis.

The early pro-inflammatory cascade can be initiated by endotoxin (also known as lipopolysaccharide, or LPS). LPS is one of the major constituents of the cell walls of gram-negative bacteria. Recognition of conserved microbial products, such as LPS, by the innate immune system leads to a variety of signal transduction pathways. These signal transduction pathways mediate the induction and secretion of cytokines that can regulate the level and duration of an inflammatory response. The systemic inflammatory response that accompanies endotoxic shock (caused by triggers such as the presence of LPS) is controlled by the levels of pro- and anti-inflammatory cytokines. Although the production of pro-inflammatory cytokines by cells of the innate immune system plays an important role in mediating the initial host defense against invading pathogens (O'Neill, 2000), an inability to regulate the nature or duration of the host's inflammatory response can often mediate detrimental host effects as observed in chronic inflammatory diseases. Additionally, in the early stages of sepsis, the host's inflammatory response is believed to be in a hyperactive state with a predominant increase in the production of pro-inflammatory cytokines that mediate host tissue injury and lethal shock (Cohen, 2002). In this regard, the ability to suppress pro-inflammatory cytokines and/or enhance anti-inflammatory cytokines, i.e. IL-10, has been shown to severely reduce the toxic effects of endotoxin (Berg, 1995; Howard, 1993).

Ghrelin is a 28 amino acid acylated polypeptide secreted predominantly from X/A-like cells of the stomach (Kojima et al. Nature. 402: 656-660 (1999)). Ghrelin has been implicated in growth hormone (GH) release, energy balance, food intake and long-term regulation of body weight in rodents (Tschop et al. Nature 407: 908-913 (2000), Nakazato et al. Nature. 409: 194-198 (2001)) and humans (Cummings et al. New Engl. J. Med. 346: 1623-1630 (2002)). The ghrelin gene encodes a 117 amino acid peptide, pre-pro-ghrelin that shares 82% homology between rat and human (Kojima et al., 1999). Ghrelin is regarded as the only known circulating orexigen and exerts antagonistic effects on the leptin-induced decrease in food intake through activation of the hypothalamic NPY/Y1 pathway (Nakazato et al. (2001), Inui, A. Ghrelin Nature Rev. Neurosci. 2: 551-560 (2001)). The effects of ghrelin are mediated via a seven transmembrane G protein coupled receptor called growth hormone secretagogue receptor GHS-R (Howard et al. Science. 273: 974-977 (1996)). This receptor is evolutionarily conserved from pufferfish to humans (Palyha et al. Mol. Endocrinol. 14: 160-169 (2000)) showing that ghrelin plays a fundamental role in organism growth and development. The GHS-R type 1a receptor has been implicated in GH release and a non-spliced, non-functional receptor mRNA variant identified as GHS-R 1b has been identified within a wide variety of tissues including lymphoid organs (Gnanapavan et al. J. Clin. Endocrinol. Metab. 87: 2988-2991 (2002)). Hexarelin is a synthetic analogue that binds GHS-R to induce GH secretion from porcine and bovine peripheral blood mononuclear cells (PBMCs) showing that GHS-R ligands can exert some direct effects on the immune system (Dantzer, R. Ann. NY. Acad. Sci. 933: 222-234 (2001)). In addition, the wide tissue distribution of GHS-R in the lymphoid system suggests that ghrelin and GHS-R ligands can function as signal modulators between the endocrine, nervous and immune system.

Inflammatory cytokines released by immune cells have been shown to act on the central nervous system (CNS) to control food intake and energy homeostasis (Hart, B L. Neurosci. Biobehav. Rev. 12: 123-137 (1988)). Decrease in food intake or anorexia is one of the most common symptoms of illness, injury or inflammation (Kotler, D. P. Ann. Internal Med. 133: 622-634 (2000)). Cytokines such as IL-1β, IL-6 and TNF-α have been implicated in wasting associated with inflammation (Ershler et al. Annu. Rev. Med. 51: 245-270 (2000)), chronic low-grade inflammation in aging (Bruunsgaard et al. Curr. Opin. Hematol. 8:131-136 (2001), McCarty, M. F. Med. Hypotheses 52: 465-477 (1999)), and atherosclerosis (Bochkov et al. Nature. 419: 77-81 (2002)). What is needed in the art is the regulation of inflammatory cytokine production by endogenous factors such as ghrelin to ameliorate a wide variety of ailments and disease conditions.

### SUMMARY OF THE INVENTION

The present invention provides a method of treating inflammation comprising administering ghrelin or a fragment thereof.

Also provided by the present invention is a method of treating loss of appetite comprising administering ghrelin or a fragment thereof.

Also provided by the present invention is a method of treating sepsis comprising administering ghrelin or a fragment thereof.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 (a-f) shows expression of functional GHS-R in human T cells. (a) Primary human T cells were labeled for GHS-R, and subcellular localization in lipid raft was visualized in resting and anti-CD3 activated cells. (b) Flow cytometric analysis of GHS-R on highly purified resting human T cells. Specific T cell labeling was abolished in presence of antibody-specific blocking peptide. T cells stained with control IgG demonstrated no specific labeling. (c) Flow analysis of GHS-R expression on highly purified (>96%) activated CD3⁺ human T cells. Staining specificity was demonstrated through the use of antibody-specific blocking peptide. (d) GHS-R mRNA is upregulated upon T cell activation as assessed using Agilent gene chip quantitation and real time RT-PCR, values are expressed as Mean ±SEM (* P<0.05). (e) Ghrelin induces intracellular calcium mobilization in cultured human T cells. T cells were stimulated with ghrelin (100 ng/ml), or SDF-1 (100 ng/ml) at 60 sec. T cells were also treated with the GHS-R antagonist, [D-Lys-3]-GHRP-6 (10⁻⁴ M), at 60 sec followed by ghrelin (100 ng/ml) at 180 sec. (f) Ghrelin causes actin-polymerization in human T cells. Cells were treated with ghrelin (100 ng/ml) and positive control SDF-1 (100 ng/ml) for 20 min and labeled for F-actin with phalloidin AF-594.

FIG. 2 (a-b) shows ghrelin receptors are expressed on human monocytes. (a) Human PBMCs were double stained with CD14 PE and GHS-R AF-488. (b) Immunofluorescence labeling revealed GHS-R expression on cell surface of purified monocytes (upper panel), negative control failed show any specific staining (lower panel).

FIG. 3 (a-h) shows ghrelin inhibits inflammatory cytokine expression from human PBMCs and T cells. Human PBMCs (n=6) were stimulated with PHA (1 μg/ml) (a-d) or T cells were activated via immobilized anti-CD3 antibody (e-h) in presence or absence of various doses of ghrelin (closed circles) and concomitantly with GHS-R antagonist, [D-Lys-3]-GHRP-6 (10⁻⁴ M; open circles) for 24 h. The harvested supernatants were subsequently assayed for IL-1β (a, e), IL-6 (b, f) and TNF-α (c, g) and TGF-β (d). The cytokine protein data is expressed as the mean ±SEM representing 6 healthy adult donors (* P<0.05). (h) Fold change in IL-1β, IL-6 and TNF-α. mRNA expression in T cells after normalization with GAPDH measured by real time RT-PCR.

FIG. 4 (a-e) shows ghrelin inhibits leptin-induced increase in inflammatory cytokines. (a) The localization of the leptin receptor, (Ob-R) on the surface of human T cells. (b-d) Anti-CD3 mAb-activated T cells from human adult donors (n=6) were incubated with various concentration of leptin or co-incubated with various doses of ghrelin with a biologically optimal concentration of leptin (100 nM). Cytokine production and mRNA expression was evaluated after 24 h of culture. The cytokines examined were (b) IL-1β, (c) IL-6, and (d) TNF-α. (e) Fold change in IL-1β, IL-6 and TNF-α. mRNA expression after normalization with GAPDH and measured by real time RT-PCR. Values are expressed as Mean ±SEM (* P<0.05).

FIG. 5 (a-g) shows ghrelin is expressed and secreted from human T cells. (a) Ghrelin and GHS-R co-expression in resting T cells (upper); Activated T cells demonstrating that ghrelin is strongly co-localized in GM1⁺ lipid rafts (middle); Pre-pro-ghrelin co-localizes in Golgi bodies in activated human T cells (lower). (b) Kinetics of ghrelin secretion from anti-CD3 mAb-stimulated T cells. (c) Fold change in ghrelin mRNA levels upon T cell activation as assessed by real time RT-PCR analysis. Values are expressed as Mean ±SEM (* P<0.05). (d) Ghrelin expression was quantitated in T cells stimulated in presence of immobilized anti CD3 antibody and in presence or absence of different concentrations of leptin after 24 h in culture. Fold change in ghrelin mRNA expression (closed bars) after normalization with GAPDH and measured by real time RT-PCR. Ghrelin protein production was determined by EIA (open bars). (e) Fold change in GHS-R gene expression after normalization with GAPDH (n=6), with values being expressed as Mean ±SEM (*p<0.05). (f) Hypothetical model for functional role of ghrelin as a signal linking immune-endocrine systems in control of food intake. (g) Comparative ghrelin mRNA expression in stomach as compared to lymphoid organs.

FIG. 6 (a-f) shows ghrelin inhibits inflammatory cytokine expression and anorexia in a murine endotoxemia model. Real time PCR analysis of inflammatory cytokine mRNA in spleen and liver 4 h and 24 h after LPS and ghrelin administration in BALB/c mice. Ct values for cytokines were normalized with GAPDH and expressed as fold change over collapsed control sham Ct values (n=6). At 4 and 24 hours post LPS injection, Ghrelin inhibits IL-1β (a,d) and IL-6 (b,e) transcription in both spleen and liver. TNF-α mRNA expression was attenuated at 4 h post LPS in spleen, but ghrelin failed to further inhibit TNF-α in spleen at 24 h. However, ghrelin continued to significantly suppress TNF-α mRNA in liver (c,f).

FIG. 7 (a-i) shows cytokine levels in the serum of treated mice after LPS and ghrelin treatment. Cytokines tested were IL-1β (a), IL-6 (b), and TNF-α (c) at 4 h and IL-1β (d), IL-6 (e) at 24 h. Ghrelin stimulates food intake in LPS challenged mice (f). Ghrelin treatment inhibits basal IL-1β and ILα secretion in periphery (g,h). Ghrelin also inhibits serum IL-1α levels 24 h post LPS challenge (i). Values are expressed as mean ±SEM (* P<0.05).

FIG. 8 (a-d) shows GHS-R expression. (a) GHS-R expression on activated purified human T cells, utilizing an antibody recognizing 186-265 amino acids near C terminal region of GHS-R peptide of human origin. (b) Differential pattern of GHS-R expression on resting human T cells. Lower panel reveals punctate GHS-R expression on resting T cells demonstrate some minor co-localization with GM-1 positive rafts. (c) Proliferation of human T cells (open circles) and IL-2 levels (closed circles) in response to anti-CD3 mAb and ghrelin treatment. Ghrelin demonstrated had no significant effects (p<0.05) on either T cell proliferation or IL-2 secretion. (d) Specificity of anti-ghrelin and anti-pre-pro ghrelin labeling in purified T cells. These images were acquired using equal exposure time and gain.

FIG. 9 shows acylated ghrelin is co-expressed with total ghrelin in human PBMCs. (a) total ghrelin was labeled with anti-rabbit antibody followed by secondary antibody conjugated with AF-594 (red) (b) acylated ghrelin expression was assessed using a anti-guinea pig antibody, and specific secondary antibody conjugated with AF-488 (green). Nuclei were stained with DAPI. (c) Merge reveals approximately 30% of the cells expressing total ghrelin also co-express the active octanoylated form of ghrelin.

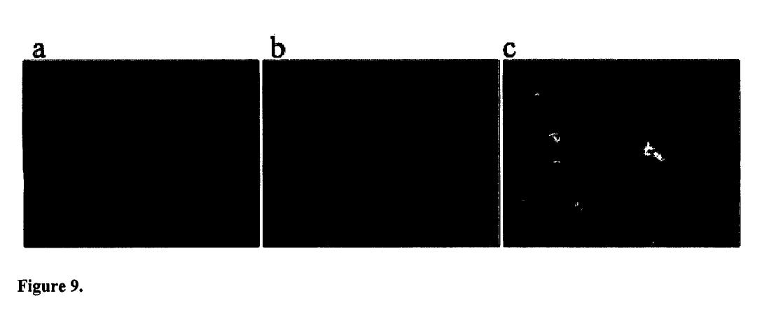

FIG. 10 shows T cell derived ghrelin is critical for homeostatic regulation of proinflammatory cytokines and chemokines. (a-b) Ghrelin expression in T cells was down-regulated using siRNA. Reduction in ghrelin levels increases the proinflammatory cytokines of human T cells.

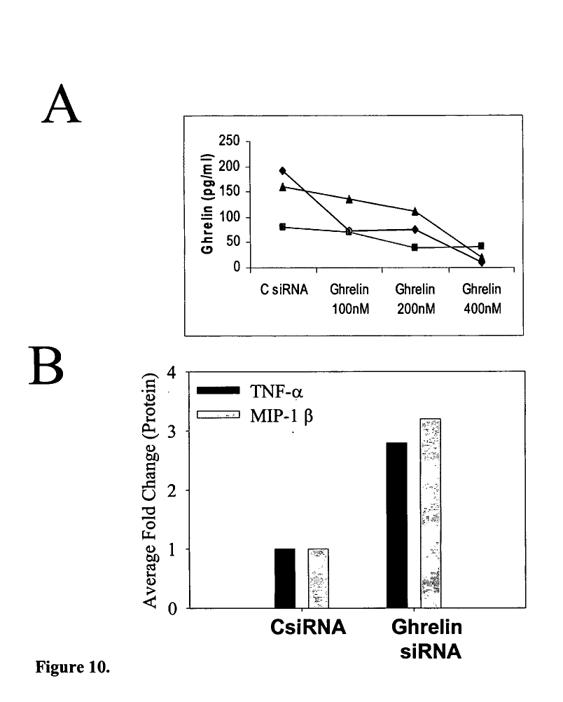

FIG. 11 shows serum ghrelin levels decline in patients with Crohn's disease.

FIG. 12 shows ghrelin expression declines in ulcerative colitis.

### DETAILED DESCRIPTION OF THE INVENTION

The present invention may be understood more readily by reference to the following detailed description of preferred embodiments of the invention and the Examples included therein and to the Figures and their previous and following description.

### A. DEFINITIONS

Before the present methods and compositions are disclosed and described, it is to be understood that this invention is not limited to specific methods or specific substances unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a substance” includes one or more substances, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The terms “higher,” “increases,” “elevates,” or “elevation” refer to increases above basal levels, or as compared to a control. The terms “low,” “lower,” “inhibits,” “inhibition,” “reduces,” or “reduction” refer to decreases below basal levels, or as compared to a control. For example, basal levels are normal in vivo levels prior to, or in the absence of, inflammation or the addition of an agent which causes inflammation.

The terms “mediate” or “mediation” and “modulate” or “modulation” mean to regulate, or control, in particular to increase, enhance, elevate, or alternatively to lower, inhibit, or reduce. The terms “mediate” and “modulate” are used interchangeably throughout.

“Inflammation” or “inflammatory” is defined as the reaction of living tissues to injury, infection, or irritation. Anything that stimulates an inflammatory response is said to be inflammatory.

“Inflammatory disease” is defined as any disease state associated with inflammation.

“Infection” or “infectious process” is defined as one organism being invaded by any type of foreign material or another organism. The results of an infection can include growth of the foreign organism, the production of toxins, and damage to the host organism. Infection includes viral, bacterial, parasitic, and fungal infections, for example.

“Liver toxicity” is defined as an abnormal accumulation of toxic substances in the liver. A number of criteria can be used to assess the clinical significance of toxicity data: (a) type/severity of injury, (b) reversibility, (c) mechanism of toxicity, (d) interspecies differences, (e) availability of sensitive biomarkers of toxicity, (e) safety margin (non toxic dose/pharmacologically active dose), and (f) therapeutic potential.

“Cancer therapy” is defined as any treatment or therapy useful in preventing, treating, or ameliorating the symptoms associated with cancer. Cancer therapy can include, but is not limited to, apoptosis induction, radiation therapy, and chemotherapy.

“Transplant” is defined as the transplantation of an organ or body part from one organism to another.

“Transplant rejection” is defined as an immune response triggered by the presence of foreign blood or tissue in the body of a subject. In one example of transplant rejection, antibodies are formed against foreign antigens on the transplanted material.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, a human.

The terms “control levels” or “control cells” are defined as the standard by which a change is measured, for example, the controls are not subjected to the experiment, but are instead subjected to a defined set of parameters, or the controls are based on pre- or post-treatment levels.

By “treating” is meant that an improvement in the disease state, i.e., the inflammatory response, is observed and/or detected upon administration of a substance of the present invention to a subject. Treatment can range from a positive change in a symptom or symptoms of the disease to complete amelioration of the inflammatory response (e.g., reduction in severity or intensity of disease, alteration of clinical parameters indicative of the subject's condition, relief of discomfort or increased or enhanced function), as detected by art-known techniques.

By “preventing” is meant that after administration of a substance of the present invention to a subject, the subject does not develop the symptoms of inflammation.

### B. COMPOSITIONS

Disclosed are the components to be used to prepare the disclosed compositions as well as the compositions themselves. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular peptide is disclosed and discussed and a number of modifications that can be made to a number of molecules including the peptide are discussed, specifically contemplated is each and every combination and permutation of the amino acids within the peptide and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Ghrelin

The term “ghrelin” is used throughout to refer to any ghrelin molecule or functional fragment thereof, as described above. The present invention includes a method of treating inflammation in a subject comprising administering to the subject an effective amount of SEQ ID NO: 1 or a fragment thereof. SEQ ID NO: 1 represents full length ghrelin (accession number AB029434). Also contemplated are methods of treating inflammation in a subject comprising administering to the subject an effective amount of SEQ ID NO: 3 (amino acids 1-18 of full length ghrelin) or a fragment thereof, SEQ ID NO: 4 (amino acids 1-14 of full length ghrelin) or a fragment thereof, SEQ ID NO: 5 (amino acids 1-10 of full length ghrelin) or a fragment thereof or SEQ ID NO: 6 (amino acids 1-5 of full length ghrelin). Also contemplated are administering fragments of any length of the above-described sequences that are functional ghrelin molecules.

Ghrelin, via functional cell surface GHS-R, exerts both specific and selective inhibitory effects on the expression and production of inflammatory cytokines such as IL-1β, IL-6 and TNF-α, by human PBMCs and T cells. The GHS-R on primary and cultured human T cells, similar to other classical GPCRs, elicits a potent intracellular calcium release upon ligation with its natural ligand, ghrelin, and is preferentially associated with GM1 lipid rafts upon cellular activation. Consistent with expression of functional GHS-R on T cells, ghrelin actively induces actin polymerization within T cells. Similar to chemokines (SDF-1), ghrelin treatment led to the cellular polarization of leukocytes and actin distribution changes from a linear cortical pattern in resting lymphocytes to more concentrated patterns at the leading edge and contact zones in polarized and activated T cells (Taub et al. Science. 260: 355-358 (1993), Inui, A Cancer Res. 59: 4493-4501 (1999)). These GPCR-like redistribution patterns show an important role for GHS-R in immune cell signaling and trafficking.

Previously, it was thought that ghrelin was only produced by endocrine-like cells in the stomach and was then released into the circulation. Through a number of analytical techniques, it has been demonstrated that ghrelin is endogenously produced and secreted by both T cells and PBMCs in a fashion similar to many immune-derived cytokines. The majority of T cells examined from human donors were found to constitutively express low levels of endogenous ghrelin, which is significantly increased upon cellular activation. Activated T cells express and secrete the ghrelin protein, exhibiting that pre-pro peptide must be actively cleaved in T cells to yield the active ghrelin peptide. Similar to several cytokines (e.g., TGF-β) and hormones (e.g., TSH), these precursor proteins are synthesized and subsequently stored for immediate cleavage and use when needed. Furthermore, the expression and secretion of a mature form of ghrelin from T cells post activation via T cell receptor ligation has been demonstrated. Given that gastrectomy results in only a 35 to 50% decline in circulating ghrelin and that ghrelin levels increase to two thirds of pre-gastrectomy levels in human subjects, it has been shown that other tissues compensate for maintaining the circulating ghrelin (Hosoda H et al J Biol Chem. 2003 Jan. 3; 278(1):64-70). Secretion of ghrelin from T cells shows that immune cell-derived ghrelin makes up part of the residual concentration of circulating ghrelin. In addition, ghrelin is also regarded as the only known hormone where the hydroxyl group of its third serine residue is acylated by n-octanoic acid and this acylation is critical for some of the biological activities of this polypeptide (Kojima et al. (1999)). N-terminal acylated peptides are known to preferentially aggregate in cholesterol rich micro-domains (Basa, et al. Neurosci. Lett. 343: 25-28 (2003)), and ghrelin is immunoreactive in activated T cells and is highly co-localized within cholesterol-rich GM1⁺ domains. These results show that ghrelin is selectively targeted to the plasma membrane to facilitate interaction with its own transmembrane receptor to optimally mediate receptor-ligand interactions. Such a pathway shows the role of ghrelin in the control of immune responses. In addition, localized production of ghrelin plays a critical role in the immediate control of ongoing and leptin-mediated responses within the local microenvironment.

2. Homology/Identity

It is understood that one way to define any known variants and derivatives or those that might arise, of the disclosed genes and proteins herein is through defining the variants and derivatives in terms of homology to specific known sequences. The term “ghrelin” is used throughout to refer to any ghrelin molecule or functional fragment thereof. “Fragment” is defined as any subpart of the reference sequence. For example SEQ ID NO:2 sets forth a particular sequence of a nucleic acid molecule encoding ghrelin, and SEQ ID NO: 1 sets forth a particular sequence of the protein encoded by SEQ ID NO: 2, the ghrelin protein. The methods of the invention include using full length ghrelin, as represented by SEQ ID NO: 1 (GenBank accession number AB029434), as well as fragments thereof. Also included are sequences longer than SEQ ID NO: 1 and include amino acids before and/or after the functional ghrelin molecule. Examples of fragments of SEQ ID NO: 1, as well as sequences longer than the functional molecule of SEQ ID NO: 1, that are useful with the methods disclosed herein include amino acids 1-5 (represented by SEQ ID NO: 6), 1-6, 1-7, 1-8, 1-9, 1-10 (represented by SEQ ID NO: 5), 1-11, 1-12, 1-13, 1-14 (represented by SEQ ID NO: 4), 1-15, 1-16, 1-17, 1-18 (represented by SEQ ID NO: 3), 1-19, 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38, 1-39, 1-40, 1-41, 1-42, 1-43, 1-44, 1-45, 1-46, 1-47, 1-48, 1-49, 1-50, 1-75, 1-100, 1-125, 1-150, 1-175, 1-200, 1-225, 1-250, 1-300, 1-350, 1-400, 1-450, and 1-500, as well as all lengths of fragments in between.

Also specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, (1989), Jaeger et al. Proc. Natl. Acad. Sci. USA 86:7706-7710 (1989), Jaeger et al. Methods Enzymol. 183:281-306 (1989) which are herein incorporated by reference for at least material related to nucleic acid alignment.

3. Nucleic Acids

There are a variety of molecules disclosed herein that are nucleic acid based, including for example the nucleic acids that encode, for example, ghrelin as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 86, 6553-6556 (1989)).

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b. Sequences

There are a variety of sequences related to, for example, ghrelin, as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

4. Peptides

a. Peptide Variants

As discussed herein there are numerous variants of the ghrelin protein that are known and herein contemplated. In addition, to the known functional ghrelin species variants there are derivatives of the ghrelin proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| alanine | Ala | A |
| arginine | Arg | R |
| asparagine | Asn | N |
| aspartic acid | Asp | D |
| cysteine | Cys | C |
| glutamic acid | Glu | E |
| glutamine | Gln | K |
| glycine | Gly | G |
| histidine | His | H |
| isolelucine | Ile | I |
| leucine | Leu | L |
| lysine | Lys | K |
| phenylalanine | Phe | F |
| proline | Pro | P |
| serine | Ser | S |
| threonine | Thr | T |
| tyrosine | Tyr | Y |
| tryptophan | Trp | W |
| valine | Val | V |

TABLE 2
Amino Acid Substitutions
Original Residue Exemplary Conservative
Substitutions, others are known in the art.

Ala and Ser
Arg and Lys, Gln
Asn and Gln, His
Asp and Glu
Cys and Ser
Gln and Asn, Lys
Glu and Asp
Gly and Pro
His and Asn, Gln
Ile and Leu, Val
Leu and Ile, Val
Lys and Arg; Gln
Met and Leu, ile
Phe and Met, Leu, Tyr
Ser and Thr
Thr and Ser
Trp and Tyr
Tyr and Trp, Phe
Val and Ile, Leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 (1983)), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NOS:1, 3, 4, 5, and 6 set forth particular sequences of ghrelin. Specifically disclosed are variants of these and other proteins herein disclosed which have at least 50%, 51%, 52%, 53%, 54%, 55%, 56%, 57%, 58%, 59%, 60%, 61%, 62%, 63%, 64%, 65%, 66%, 67%, 68%, 69%, 70%, 71% 72%, 73%, 74%, 75%, 76%, 77%, 78%, 79%, 80% 81%, 82%, 83%, 84%, 85%, 86%, 87%, 88%, 89%, 90%, 91%, 92%, 93%, 94%, 95%, 96%, 97%, 98%, or 99% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman (Adv. Appl. Math. 2: 482 (1981), by the homology alignment algorithm of Needleman and Wunsch (J. Mol. Biol. 48: 443 (1970)), by the search for similarity method of Pearson and Lipman (Proc. Natl. Acad. Sci. U.S.A. 85: 2444 (1988)), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52 (1989), Jaeger et al. Proc. Natl. Acad. Sci. USA 86:7706-7710 (1989), Jaeger et al. Methods Enzymol. 183:281-306 (1989) which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:1 is set forth in SEQ ID NO:2. It is understood that while no amino acid sequence indicates what particular DNA sequence encodes that protein within an organism, where particular variants of a disclosed protein are disclosed herein, the known nucleic acid sequence that encodes that protein in the particular sequence from which that protein arises is also known and herein disclosed and described.

It is understood that there are numerous amino acid and peptide analogs which can be incorporated into the disclosed compositions. For example, there are numerous D amino acids or amino acids which have a different functional substituent than the amino acids shown in Table 1 and Table 2. The opposite stereoisomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994)) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data, Vol. 1, Issue 3, Peptide Backbone Modifications (general review) (March 1983); Morley, Trends Pharm Sci pp. 463-468 (1980); Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### C. METHODS OF TREATMENT AND PREVENTION

1. Inflammation

The present invention provides a method of treating inflammation in a subject comprising administering to the subject an effective amount of ghrelin. Inflammation can be associated with a number of different diseases and disorders. Examples of inflammation include, but are not limited to, inflammation associated with hepatitis, inflammation associated with the lungs, inflammation associated with burns, and inflammation associated with an infectious process. Inflammation can also be associated with liver toxicity, which can be associated in turn with cancer therapy, such as apoptosis induction or chemotherapy, or a combination of the two, for example.

Inhibition of the NFkB pathway has been identified as one of the major mediators of ghrelin's protective effects (Example 8). NFkB regulatory genes regulated by ghrelin were identified as TRCP, TOM1, AP2, GAB1 and TANK. Therefore, disclosed are methods of treating inflammation comprising targeting TRCP, TOM1, AP2, GAB1 and TANK with ghrelin.

The inflammation can be associated with an inflammatory disease. Examples of inflammatory disease include, but are not limited to, asthma, systemic lupus erythematosus, rheumatoid arthritis, reactive arthritis, spondyarthritis, systemic vasculitis, insulin dependent diabetes mellitus, multiple sclerosis, experimental allergic encephalomyelitis, Sjögren's syndrome, graft versus host disease, inflammatory bowel disease including Crohn's disease, ulcerative colitis, and scleroderma. Inflammatory diseases also includes autoimmune diseases such as myasthenia gravis, Guillain-Barré disease, primary biliary cirrhosis, hepatitis, hemolytic anemia, uveitis, Grave's disease, pernicious anemia, thrombocytopenia, Hashimoto's thyroiditis, oophoritis, orchitis, adrenal gland diseases, anti-phospholipid syndrome, Wegener's granulomatosis, Behcet's disease, polymyositis, dermatomyositis, multiple sclerosis, vitiligo, ankylosing spondylitis, Pemphigus vulgaris, psoriasis, dermatitis herpetiformis, Addison's disease, Goodpasture's syndrome, Basedow's disease, thrombopenia purpura, allergy, and cardiomyopathy.

The inflammation can also be associated with cancer. Examples of types of cancer include, but are not limited to, lymphoma (Hodgkins and non-Hodgkins) B-cell lymphoma, T-cell lymphoma, leukemia such as myeloid leukemia and other types of leukemia, mycosis fungoide, carcinoma, adenocarcinoma, sarcoma, glioma, blastoma, neuroblastoma, plasmacytoma, histiocytoma, melanoma, adenoma, hypoxic tumour, myeloma, AIDS-related lymphoma or AIDS-related sarcoma, metastatic cancer, bladder cancer, brain cancer, nervous system cancer, squamous cell carcinoma of the head and neck, neuroblastoma, glioblastoma, ovarian cancer, skin cancer, liver cancer, squamous cell carcinomas of the mouth, throat, larynx, and lung, colon cancer, cervical cancer, breast cancer, cervical carcinoma, epithelial cancer, renal cancer, genitourinary cancer, pulmonary cancer, esophageal carcinoma, head and neck carcinoma, hematopoietic cancer, testicular cancer, colo-rectal cancer, prostatic cancer, and pancreatic cancer.

Activated cells can also be treated at the site of inflammation. “Activated cells” are defined as cells that participate in the inflammatory response. Examples of such cells include, but are not limited to, T-cells and B-cells, macrophages, NK cells, mast cells, eosinophils, neutrophils, Kupffer cells, antigen presenting cells, as well as vascular endothelial cells.

2. Infection

Inflammation can be caused by an infectious process in a subject. When the inflammation is associated with an infectious process, the infectious process can be associated with a viral infection. Examples of viral infections include, but are not limited to, Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian immunodeficiency cirus, Human T-cell Leukemia virus type-1, Hantavirus, Rubella virus, Simian immunodeficiency virus, Human Immunodeficiency virus type-1, and Human immunodeficiency virus type-2.

When the inflammation is associated with an infectious process, the infectious process can be associated with a bacterial infection. The bacterial infection can be caused by either gram positive or gram negative bacterium. The gram positive bacterium can be selected from the group consisting of: M. tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israeli and other Actinomyces species, and Propionibacterium acnes.

The gram negative bacterium can be selected from the group consisting of: Clostridium tetani, Clostridium perfringens, Clostridium botulinum, other Clostridium species, Pseudomonas aeruginosa, other Pseudomonas species, Campylobacter species, Vibrio cholerae, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteriacae, Brucella abortus and other Brucella species, Burkholderia cepacia, Burkholderia pseudomallei, Francisella tularensis, Bacteroides fragilis, Fusobascterium nucleatum, Provetella species and Cowdria ruminantium.

The above examples of gram positive and gram negative bacteria are not intended to be limiting, but are intended to be representative of a larger population including all gram positive and gram negative bacteria, as well as non-gram test responsive bacteria. Examples of other species of bacteria include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella.

When the inflammation is associated with an infectious process, the infectious process can be associated with a parasitic infection. Examples of parasitic infections include, but are not limited to, Toxoplasma gondii, Plasmodium species such as Plasm odium falciparum, Plasm odium vivax, Plasmodium malariae, and other Plasmodium species, Trypanosoma brucei, Trypanosoma cruzi, Leishmania species such as Leishmania major, Schistosoma such as Schistosoma mansoni and other Shistosoma species, and Entamoeba histolytica.

When the inflammation is associated with an infectious process, the infectious process can be associated with a fungal infection. Examples of fungal infections include, but are not limited to, Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata.

3. Sepsis

Furthermore, the infection can be associated with sepsis. Sepsis, also known as systemic inflammatory response syndrome (SIRS), is a severe illness caused by overwhelming infection of the bloodstream by toxin-producing bacteria. Sepsis occurs in 2 of every 100 hospital admissions. It is caused by bacterial infection that can originate anywhere in the body. Common sites include, but are not limited to, the kidneys (upper urinary tract infection), the liver or the gall bladder, the bowel (usually seen with peritonitis), the skin (cellulitis), and the lungs (bacterial pneumonia).

LPS-induced endotoxemia in mice is a well recognized model for inducing septic shock and is also associated with anorexia due to excessive production of pro-inflammatory mediators. In spite of a large body of data, the causes of systemic inflammatory response syndrome (SIRS) remain unknown and various therapeutic approaches have yielded minimally beneficial results (Riedemann et al. J. Clin. Invest. 112: 460-467 (2003), Luheshi et al. Proc. Natl. Acad. Sci. USA. 96: 7047-7052 (1999)). LPS directly acts on mononuclear cells, but the resultant endotoxemia also affects a wide variety of cells and systems and is associated with a refractory catabolic state.

It was demonstrated that ghrelin infusions in LPS challenged mice led to a significant inhibition of pro-inflammatory cytokines IL-1α and β, IL-6 and TNF-α in circulation as well as in liver, spleen, lungs and mesenteric lymph nodes. In addition, LPS-induced endotoxemia resulted in inhibition of ghrelin secretion (Hataya et al. Endocrinology. 144: 5365-5371 (2003)), and ghrelin infusion increases body weight in septic animals (Murray et al. Gastroenterology. 125: 1492-1502 (2003)). Therefore, inhibition of ghrelin secretion, post-LPS challenge, exacerbates the ongoing inflammatory insult and promotes development of a catabolic state. Furthermore, it was demonstrated that LPS induced inflammatory anorexia is also significantly reduced in ghrelin treated mice. The inclusion of ghrelin and synthetic GHS are therefore candidates in treatment of SIRS. Ghrelin also plays a regulatory role in chronic conditions such as Helicobacter pylori infection where persisting gastric inflammation is associated with lower ghrelin levels (49) and correction of infection leads to up regulation of ghrelin secretion.

Meningitis may also be accompanied by sepsis. In children, sepsis may accompany infection of the bone (osteomyelitis). In hospitalized patients, common sites of infection include intravenous lines, surgical wounds, surgical drains, and sites of skin breakdown known as decubitus ulcers or bedsores. The infection is often confirmed by a positive blood culture, though blood cultures may be negative in individuals who have been receiving antibiotics. In sepsis, blood pressure drops, resulting in shock. Major organs and systems, including the kidneys, liver, lungs, and central nervous system, stop functioning normally. Sepsis is often life-threatening, especially in people with a weakened immune system or other medical illnesses.

4. Transplantation

Inflammation can be associated with transplant rejection in a transplant or implant recipient. As disclosed, above, “transplant rejection” is defined as an immune response triggered by the presence of foreign blood or tissue in the body of a subject. In one example of transplant rejection, antibodies are formed against foreign antigens on the transplanted material. The transplantation can be, for example, tissue, cell or organ transplantation, such as liver, kidney, skin, corneal, pancreas, pancreatic islet cells, eyes, heart, or any other transplantable organ of the body.

Transplantation immunology refers to an extensive sequence of events that occurs after an allograft or a xenograft is removed from a donor and then transplanted into a recipient. Tissue is damaged at both the graft and the transplantation sites. An inflammatory reaction follows immediately, as does activation of biochemical cascades. Such an inflammatory reaction can be reduced using the methods taught herein. In the inflammatory reaction, a series of specific and nonspecific cellular responses ensues as antigens are recognized. Antigen-independent causes of tissue damage (i.e., ischemia, hypothermia, reperfusion injury) are the result of mechanical trauma as well as disruption of the blood supply as the graft is harvested. In contrast, antigen-dependent causes of tissue damage involve immune-mediated damage.

Macrophages release cytokines (e.g., tumor necrosis factor, interleukin-1), which heighten the intensity of inflammation by stimulating inflammatory endothelial responses; these endothelial changes help recruit large numbers of T cells to the transplantation site.

Damaged tissues release pro-inflammatory mediators (e.g., Hageman factor (factor XII) that trigger several biochemical cascades. The clotting cascade induces fibrin and several related fibrinopeptides, which promote local vascular permeability and attract neutrophils and macrophages. The kinin cascade principally produces bradykinin, which promotes vasodilation, smooth muscle contraction, and increased vascular permeability.

Rejection is the consequence of the recipient's alloimmune response to the nonself antigens expressed by donor tissues. In hyperacute rejection, transplant subjects are serologically presensitized to alloantigens (i.e., graft antigens are recognized as nonself). Histologically, numerous polymorphonuclear leukocytes (PMNs) exist within the graft vasculature and are associated with widespread microthrombin formation and platelet accumulation. Little or no leukocyte infiltration occurs. Hyperacute rejection manifests within minutes to hours of graft implantation. Hyperacute rejection has become relatively rare since the introduction of routine pretransplantation screening of graft recipients for antidonor antibodies.

In acute rejection, graft antigens are recognized by T cells; the resulting cytokine release eventually leads to tissue distortion, vascular insufficiency, and cell destruction. Histologically, leukocytes are present, dominated by equivalent numbers of macrophages and T cells within the interstitium. These processes can occur within 24 hours of transplantation and occur over a period of days to weeks.

In chronic rejection, pathologic tissue remodeling results from peritransplant and posttransplant trauma. Cytokines and tissue growth factor induce smooth muscle cells to proliferate, to migrate, and to produce new matrix material. Interstitial fibroblasts are also induced to produce collagen. Histologically, progressive neointimal formation occurs within large and medium arteries and, to a lesser extent, within veins of the graft. Leukocyte infiltration usually is mild or even absent. All these result in reduced blood flow, with subsequent regional tissue ischemia, fibrosis, and cell death. (Prescilla et al. emedicine website, Immunology of Transplant Rejection, updated Jun. 20, 2003).

Transplant rejection may occur within 1-10 minutes of transplantation, or within 10 minutes to 1 hour of transplantation, or within 1 hour to 10 hours of transplantation, or within 10 hours to 24 hours of transplantation, within 24 hours to 48 hours of transplantation, within 48 hours to 1 month of transplantation, within 1 month to 1 year of transplantation, within 1 year to 5 years of transplantation, or even longer after transplantation.

Any animal which is subject to inflammation can be treated by this method. Therefore, the subject can be any mammal, preferably human, and can include but is not limited to mouse, rat, cow, guinea pig, hamster, rabbit, cat, dog, goat, sheep, monkey, horse and chimpanzee.

5. Loss of Appetite

The present invention provides a method of treating loss of appetite in a subject by administering to the subject an effective amount of ghrelin. Loss of appetite can be caused by a wide variety of substances, diseases and disorders. Examples of such include, but are not limited to, emotional upset, nervousness, loneliness, tension, anxiety, bereavement, depression, anorexia nervosa, anorexia-cachexia syndrome, acute and chronic infections (as described above), HIV, pregnancy, cancer, atherosclerosis, inflammation (both acute and chronic, as well as low grade inflammation), hyperthyroidism, medications and street drugs, chemotherapeutic agents, amphetamines, sympathomimetics including ephedrine, antibiotics, cough and cold preparations, codeine, morphine, demerol, and digitalis. As shown in Example 7, ghrelin treatment resulted in a significant attenuation of LPS-induced anorexia as well as increased the appetites of non-LPS treated mice.

Low-grade inflammation can be associated with aging. as aging is associated with an increase in inflammatory cytokines including IL-6. The increase in inflammatory mediators with age is related to ‘anorexia of aging’ and fraility (Ershler, W. B., and Keller, T. E. 2000. Age-associated increased interleukin-6 gene expression, late life diseases, and frailty. Annu. Rev. Med. 51: 245-270). Ghrelin supplementation therapy of frail and aging subjects can reduce the ongoing inflammatory insult, increase food intake and promote the anabolic processes.

6. Cytokines

Also disclosed are methods of inhibiting secretion of cytokines, comprising administering an effective amount of ghrelin. For example, the cytokines can be inhibited at the site of inflammation. The cytokine can be expressed by cells selected from the group consisting of T-cells, B-cells, dendritic cells, and mononuclear cells.

Examples of cytokines and immunomodulatory agents that can be employed in the methods of this invention include, but are not limited to, those participating in humoral inflammation, such as IL-3, IL-4, IL-5, IL-6, IL-7, IL-9, IL-10, IL-13, and transforming growth factor-β (TGF-β), and those contributing to cellular inflammation such as IL-1, IL-2, IL-3, IL-4, IL-7, IL-9, IL-10, IL-12, interferons (IFNs), IFN-γ inducing factor (IGIF), TGF-β and TNF-α and -β. Ghrelin can be used to modulate cytokines and/or immunomodulators according to the methods of this invention both to treat an acute episode of disease and/or to maintain the subject's condition in a non-inflammatory state.

Cytokines are proteins made by cells that affect the behavior of other cells. Cytokines made by lymphocytes are often called lymphokines or interleukins (IL). Cytokines act on specific cytokine receptors on the cells they affect. Binding of the receptor induces activity in the cell such as growth, differentiation, or death. Several cytokines play key roles in mediating acute inflammatory reactions, namely IL-1, TNF-α, IL-6, IL-1β, IL-8 and other chemokines, GCSF, and GM-CSF. Of these, IL-1 (α and β) and TNF are extremely potent inflammatory molecules: they are the primary cytokines that mediate acute inflammation induced in animals by intradermal injection of bacterial lipopolysaccharide and two of the primary mediators of septic shock.

Chronic inflammation may develop following acute inflammation and may last for weeks or months, and in some instances for years. During this phase of inflammation, cytokine interactions result in monocyte chemotaxis to the site of inflammation where macrophage activating factors (MAF), such as IFN-γ, MCP-1, and other molecules then activate the macrophages while migration inhibition factors (MIF), such as GM-CSF and IFN-γ, retain them at the inflammatory site. The macrophages contribute to the inflammatory process by chronically elaborating low levels of IL-1 and TNF which are responsible for some of the resulting clinical symptoms such as anorexia, cachexia, fever, sleepiness, and leukocytosis. The cytokines known to mediate chronic inflammatory processes can be divided into those participating in humoral inflammation, such as IL-3, IL-4, IL-5, IL-6, IL-7, IL-9, IL-10, IL-13, and transforming growth factor-β (TGF-β), and those contributing to cellular inflammation such as IL-1, IL-2, IL-3, IL-4, IL-7, IL-9, IL-10, IL-12, interferons (IFNs), IFN-γ inducing factor (IGIF), TGF-β and TNF-α and -β (Feghali et al. Frontiers in Bioscience 2, d12-26 (Jan. 1, 1997)).

The production of pro-inflammatory cytokines by cells of the innate immune system plays an important role in mediating the initial host defense against invading pathogens. Furthermore, the inability to regulate the nature or duration of the host's inflammatory response can often mediate detrimental host effects as observed in chronic inflammatory diseases. For example, in the early stages of sepsis, the host's inflammatory response is believed to be in a hyperactive state with a predominant increase in the production of pro-inflammatory cytokines that mediate host tissue injury and lethal shock. Thus, the ability of the innate immune system to dictate the levels of pro- and anti-inflammatory cytokine production is critical in limiting or modulating the nature of the host inflammatory response.

The immune system, in particular the production of inflammatory cytokines by leukocytes, plays an important role in the development of anorexia-cachexia syndrome (Hart et al. (1988), Kotler et al. (2000), Ershler et al. (2000)). Examples of cytokines considered to be relevant to inflammatory anorexia include IL-1β, IL-6 and TNF-α. Peripherally administered ghrelin is shown herein to block IL-1β-induced anorexia and produces positive energy balance by promoting food intake and decreasing energy expenditure. The inhibitory effect of ghrelin on pro-inflammatory cytokine expression shows a regulatory role for ghrelin and GHS-R in controlling cytokine-induced anorexia. Moreover, the combination of IL-1β and leptin has also been shown to inhibit ghrelin expression in stomach (Cohen, J Nature 420: 885-891 (2003)) and stomach ghrelin expression is increased in leptin deficient mice. Leptin and ghrelin are considered to exert mutually antagonistic effects on the food intake at the hypothalamic level (Nakazato et al. (2001), Inui, A. (2001)). Leptin, a member of gp130 family of cytokines, induces a strong Th1 response (Hosoda et al. J. Biol. Chem. 278: 64-70 (2003)) and is regarded as a pro-inflammatory inducer (Loffreda, S. et al. FASEB J. 12: 57-65 (1998); Zarkesh-Esfahani et al. J. Immunol. 167: 4593-4599 (2001), Lord et al. Nature. 394: 897-901 (1998), Hosoda et al. J. Biol. Chem. 278: 64-70 (2003), Dixit et al. Endocrinology. 144: 5595-5603 (2003)). Leptin's actions on food intake are controlled, in part, by an increase in the level of IL-1β in the hypothalamus (Janik et al. J. Clin. Endocrinol. Metab. 82: 3084-3086 (1997)). Similarly, anorectic effects of IL-1 are mediated via increasing leptin levels (Lambert et al. Proc. Natl. Acad. Sci. USA. 98: 4652-4657 (2001)).

It has been demonstrated that leptin can directly induce the mRNA expression and secretion of IL-1β, IL-6 and TNF-α by human T cells and PBMCs. Leptin and several other gp130 ligands including LIF, CNTF and IL-6 exert similar effects on host metabolism (Beretta et al. Peptides. 23: 975-984 (2002), Wallenius et al. Nature Med. 8: 75-79 (2002)). Moreover, IL-6^{-/-} deficient mice in a fashion similar to leptin deficient mice develop obesity (Laviano et al. Nutrition. 18: 100-105 (2002)). While leptin has been shown to be associated with cachexia, leptin levels are not elevated in many cancer-associated wasting conditions (Doehner et al. Eur. J. Endocrinol. 145: 727-735 (2001)), most likely due to a systemic decline in adipose tissue. However, cachexia seen in chronic heart failure patients is associated with hyperleptinemia (Nagaya, N. et al. Circulation. 104: 1430-1435 (2001)). In contrast, ghrelin attenuates cachexia associated with chronic heart failure in rats (Van den Berghe et al. J. Clin. Endocrinol. Metab. 84: 1311-1323 (1999)) and the GHS-R analogue, GHRP-2, counteracts protein hypercatabolism, skeletal muscle proteolysis, and osteoporosis in critically ill patients with wasting condition (Sanna et al. J. Clin. Invest. 111: 241-250 (2003)). Furthermore, increased levels of circulating leptin within a murine multiple sclerosis (MS) model regulate inflammatory anorexia and disease susceptibility (Sun, Y. et al. Mol. Cell. Biol. 23: 7973-7981 (2003)). Fasting induced suppression of leptin levels dramatically attenuates the onset of EAE in this model (Sun, Y. et al. (2003)). Not only is fasting associated with a decrease in serum leptin and a strong increase in circulating ghrelin levels (Cummings et al. (2002), Inui, A. (2001)), the observed anti-inflammatory effects of fasting in this murine MS model are also mediated by ghrelin. Given that regulation of hunger is most critical for the survival of species, a complex circuitry of compensatory mechanisms has evolved to protect against lack of one or more of these regulators.

Ghrelin functions as a vital counter-regulatory signal in the immune system controlling not only activation-induced cytokine expression but also leptin-induced expression of these same inflammatory mediators. The reciprocal regulatory effects of these hormones on expression of IL-1β, IL-6 and TNF-α by immune cells has widespread implications in the development of wasting diseases, aging, and frailty. Proposed interventions to lower ghrelin levels or blocking GHS-R for treatment of obesity can result in a potentiation of ongoing inflammatory insults or lead to immune dysregulation. On the contrary, the novel anti-inflammatory actions of ghrelin within the immune system have benefits in management of anorexia-cachexia syndrome associated with a wide range of inflammatory conditions and cancer.

7. Treatment

The agents and methods disclosed herein are of benefit to subjects who are experiencing inflammation or are at risk for inflammation, and subjects who are experiencing loss of appetite. Because the agents and methods disclosed herein reduce the severity or duration of inflammation, any subject that can benefit from a reduction in inflammation can be treated with the methods and agents disclosed herein.

The compositions comprising an agent disclosed herein in a pharmaceutically acceptable carrier may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein in its entirety for the methods taught.

The compositions may be in solution or in suspension (for example, incorporated into microparticles, liposomes, or cells). These compositions may be targeted to a particular cell type via antibodies, receptors, or receptor ligands. The following references are examples of the use of this technology to target specific proteins to given tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a. Pharmaceutically Acceptable Carriers

Administration of ghrelin or fragments thereof disclosed herein can occur in conjunction with other therapeutic agents. Thus, the agents of the present invention can be administered alone or in combination with one or more therapeutic agents. For example, a subject can be treated with the disclosed agent alone, or in combination with chemotherapeutic agents, antibodies, antivirals, steroidal and non-steroidal anti-inflammatories, conventional immunotherapeutic agents, cytokines, chemokines, and/or growth factors. Combinations may be administered either concomitantly (e.g., as an admixture), separately but simultaneously (e.g., via separate intravenous lines into the same subject), or sequentially (e.g., one of the compounds or agents is given first followed by the second). Thus, the term “combination” or “combined” is used to refer to either concomitant, simultaneous, or sequential administration of two or more agents.

Delivery of the agents disclosed herein can be used therapeutically in combination with a pharmaceutically acceptable carrier. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including opthamalically, vaginally, rectally, intranasally), orally, by inhalation, or parenterally, for example by intravenous drip, subcutaneous, intraperitoneal or intramuscular injection. The disclosed compounds can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b. Dosages

The substances of the present invention can be delivered at effective amounts or concentrations. An effective concentration or amount of a substance is one that results in treatment or prevention of the inflammatory response or loss of appetite. One skilled in the art would know how to determine an effective concentration or amount according to methods known in the art, as well as provided herein. One of skill in the art can utilize in vitro assays to optimize the in vivo dosage of a particular substance, including concentration and time course of administration.

The dosage ranges for the administration of the substances are those large enough to produce the desired effect in which the symptoms of the disorder are affected. For example, the dosage range can be from 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mg/kg body weight of ghrelin, for example, or any amount in between. In particular, the amount of ghrelin that can be administered can be about 0.5 mg/kg body weight or 1-15 mg/kg.

The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any contraindications. Dosage can vary, and can be administered in one or more dose administrations daily, for one or several days.

For example, to evaluate the efficacy of treatment of humans with a disorder characterized by inflammation with a substance that modulates cytokine activity, the following studies can be performed. Patients with active inflammation of, for example, the lung who have failed standard medical therapy, which can include prednisone and/or other immunomodulators known in the art (parenterally or orally) for control of the disorder can be selected. Drug efficacy can be monitored. Patients can be randomized to two different protocols. In one protocol, subjects can remain on initial medication and in the second protocol, subjects can have their medication tapered after receiving the substance that modulates cytokine activity, such as ghrelin.

In one example, the ghrelin can be infused over a two hour period or a weekly dosage of about 0.5 mg/kg of body weight infused each time over a two hour period until symptoms of inflammation or loss of appetite subside. The blood pressure, pulse and temperature of the subjects can be monitored prior to and at 30 minute intervals during the two hour infusion period. Subjects can also undergo routine inflammatory monitoring.

As described above, the agents disclosed herein can be administered together with other forms of therapy. For example, the molecules can be administered with antibodies, antibiotics, or other cancer treatment protocols as described above, or viral vectors. When the agent is in a vector, as described above, the vector containing the nucleic acid for therapeutic purposes can also contain ghrelin or a fragment thereof.

c. Nucleic Acid Approaches for Delivery

The substances of the present invention, including ghrelin, can also be administered in vivo and/or ex vivo to patients or subjects as a nucleic acid preparation (e.g., DNA or RNA) that encodes a substance, such as ghrelin, such that the patient's or subject's own cells take up the nucleic acid and produce and secrete the encoded substances.

The nucleic acids of the present invention can be in the form of naked DNA or RNA, or the nucleic acids can be in a vector for delivering the nucleic acids to the cells, whereby the DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, (1986)). The recombinant retrovirus can then be used to infect and thereby deliver to the infected cells nucleic acid encoding a broadly neutralizing antibody (or active fragment thereof) of the invention. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, (994)), adeno-associated viral (AAV) vectors (Goodman et al., Blood 84:1492-1500 (1994)), lentiviral vectors (Naidini et al., Science 272:263-267 (1996)), and pseudotyped retroviral vectors (Agrawal et al., Exper. Hematol. 24:738-747 (1996)). Physical transduction techniques can also be used, such as liposome delivery and receptor-mediated and other endocytosis mechanisms (see, for example, Schwartzenberger et al., Blood 87:472-478, (1996)) to name a few examples. This invention can be used in conjunction with any of these or other commonly used gene transfer methods.

As one example, if the antibody-encoding nucleic acid of the invention is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001 (1997); Alvarez and Curiel, Hum. Gene Ther. 8:597-613, (1997)). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector of the present invention, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. (1995)).

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLES

### Example 1

### General Methods

Human Subjects. Pheresis packs were prepared from 6 healthy male donors between 22-37 years age for the isolation of PBMCs and T cells.

Mice. Male 20-22 g BALB/c mice (Taconic, Germantown, N.Y.), 8-10 weeks old, were used. The guidelines proposed by the committee for the Care of Laboratory Animal Resources Commission of Life Sciences-National Research Council were followed to minimize animal pain and distress. Each animal received rodent laboratory chow and ad libitum water.

LPS-induced inflammation. Endotoxin shock in mice was induced by intraperitoneal (i.p.) injection with 10 μg of LPS (E. coli serotype 055:B5, Sigma) as described previously (Bochkov et al. 2002. Nature. 419: 77-8). Animals also received a single i.p. injection of ghrelin (5 mg/kg body weight) in PBS at 24 h and 30 min prior to LPS administration. Mice were sacrificed 4 h and 24 h post-LPS challenge and visceral organs and serum were collected.

T cell isolation and culture. Peripheral blood mononuclear cells (PBMC) were obtained by Ficoll-Hypaque density centrifugation. T cells were purified from PBMC using human T cell enrichment columns (R&D systems) via high affinity negative selection according to manufacturer's instructions. Flow analysis typically revealed greater than 90% purity. T cells were stimulated with plate bound anti-human CD3 antibody (BD Pharmingen, San Diego, Calif.) (200 ng/ml) at a concentration of 3×10⁶ cells/ml in AIM-V serum free media for 24 h.

Immunofluorescence staining. Cellular staining was performed as described previously (17). Briefly, cells were incubated with different combinations of human anti-GHS-R goat IgG, anti-GHS-R rabbit IgG recognizing 186-202 amino acids near the C terminus of human GHS-R (Santa Cruz Biotech, Santa Cruz, Calif.), anti-ghrelin rabbit IgG, anti-pre-pro-ghrelin rabbit IgG (Phoenix peptides, Belmont, Calif.) overnight at 4° C. Lipid raft were visualized using cholera toxin-Alexa fluor (AF) 594 (Molecular Probes, Eugene, Oreg.) at 20 μg/ml for 45 min. Golgi bodies were stained with goat anti-mouse Golgin-97, a marker for Golgi bodies (Molecular Probes, Eugene, Oreg.). Cells were thereafter labeled with appropriate secondary antibodies conjugated to AF-488, and AF-594. Nuclei were counter-stained using 4′, 6-diaminodino-2-phenylindole dihydrochloride (DAPI) (1 μg/ml). Images were acquired by Spot Advanced software on a Zeiss Axiovert S100 microscope under 100× objective (Carl Zeiss, Thornwood, N.Y.).

Flow Cytometric Analysis. Human PBMCs (1×10⁶) in PBS containing 2% heat-inactivated FBS were fixed using 1% paraformaldehyde and stained for CD3, CD4, CD8 PE, and CD14 PE conjugated antibodies (BD Pharmingen, San Diego, Calif.) and incubated for 30 minutes on ice. Cells were washed with PBS, and then stained for GHS-R and ghrelin followed with specific secondary antibodies conjugated to AF-488 and analyzed on FACScan.

Intracellular calcium mobilization. Measurement of intracellular calcium release in response to ghrelin and SDF-1 was performed as described previously (Sherman-Baust et al. Cancer Cell. 4: 377-386 (2003)). Cells were incubated in PBS containing 5 μM Fura-2 AM for 30 minutes at room temperature. The cells were subsequently washed and then resuspended at 1×10⁶/mL in PBS. A total of 2 mL of the cell suspension was placed in a continuously stirring cuvette at room temperature in an LS50B spectrophotometer (Perkin-Elmer, Wellesley, Mass.). Fluorescence was monitored at λₑₓ₁=340 nm, λₑₓ₂=380 nm, and λₑₘ=510 nm. The data are presented as the relative ratio of fluorescence excited at 340 and 380 nm.

Actin Polymerization. Human T cells were incubated either with ghrelin (100 ng/ml), or positive control SDF-1 (100 ng/ml) for 20 min. Thereafter cells were fixed and permeabilized in 2% Paraformaldehyde plus 0.1% Triton-X 100 and stained for actin using phalloidin AF-594 and nucleus by DAPI.

Cytokine estimation. IL-1β, IL-6 and TNF-α were estimated in T cell supernatants after 24 h using commercial ELISA kits according to manufacturer's instructions (Biosource, Camarillo, Calif.). Serum cytokines were analyzed using Bio-Plex Mouse Cytokine 18-Plex Panel according to manufacturer's instructions (Biorad, Hercules, Calif.).

Real Time RT-PCR analysis. RT-PCR was performed as described previously (Nagasawa et al. Adv. Immunol. 71: 211-228 (1999)). Total RNA (2 μg) and oligo-dT primers were used to synthesize single-stranded cDNA using the Reverse Transcription kit (Life Technologies, Gaithersburg, Md.) according to manufacturer's instructions. The PCR was set up using SYBR green Master Mix (Applied Biosystems), 1 μl cDNA and gene-specific primers at a final concentration of 0.3 μM. Thermal cycling was carried out on the Applied Biosystems GeneAmp 7700 Sequence Detector and SYBR green dye intensity was analyzed using GeneAmp 7700 SDS software. Primers for human IL-1β, IL-6, TNF-α genes and glyceraldehyde-3-phosphate dehydrogenase (GAPDH) as control were purchased from Biosource International, Camarillo, Calif., human GHS-R 1a and ghrelin were used as described previously (Gnanapavan et al. (2002)). Mouse IL-1β, IL-6, TNF-α, GAPDH and human GHS-R1a primers were designed using ABI prism software (PE Applied Biosystems). The PCR product of the GHS-R 1a amplification was quantitated using the Agilent 2100 Bioanalyzer (Agilent Technologies, Palo Alto, Calif.). Primers are available upon request. No PCR products were generated from genomic versus cDNA template.

Statistical Analysis. Results were expressed as the mean ±SEM. Statistical analysis was carried out by one-ANOVA. Significant differences between treatment groups were determined by the Student-Newman-Keuls test, statistical significance was inferred at P<0.05.

### Example 2

### GHS-R is a Functional Receptor Expressed on the Surface of Human T Cells

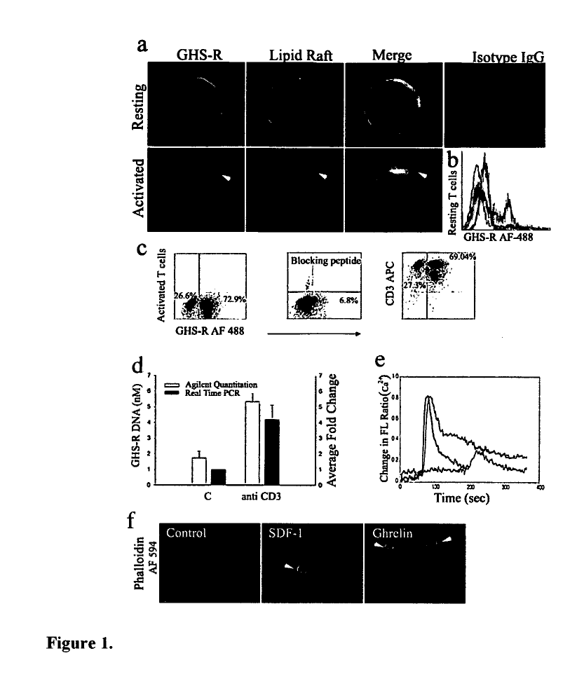

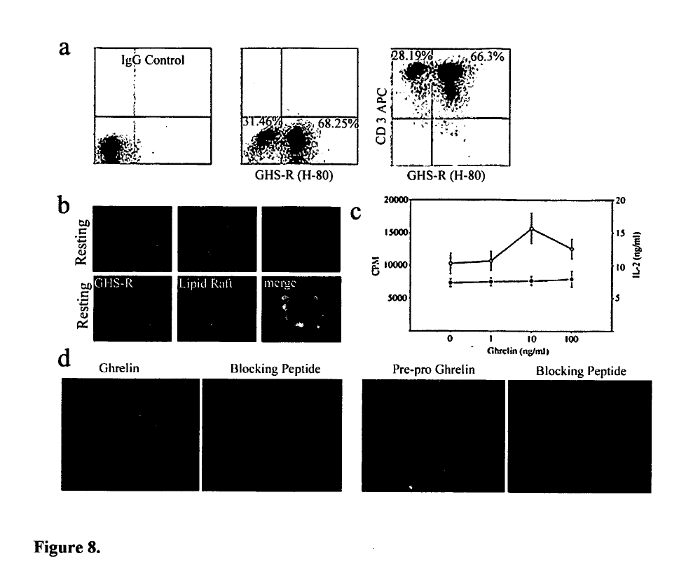

While previous results have only described the mRNA expression of GHS-R in lymphoid organs, the present studies focused on the expression and spatial localization of GHS-R protein in purified human T cells. GHS-R displays a heterogeneous subcellular expression pattern in resting human T cells ranging from crescent, punctate or diffuse phenotypes (FIG. 1a upper, FIG. 8b). In resting T cells, the majority of ghrelin receptors are segregated from the GM1+lipid rafts (FIG. 1a upper). However, upon activation of T cells via TCR ligation, a dramatic subcellular reorganization of GHS-R is observed, demonstrating a polarized capped phenotype and aggregation in lipid rafts (FIG. 1a lower). Flow cytometric analysis revealed that up to 30% of highly purified resting human T cells demonstrate specific staining for GHS-R as demonstrated via the use of a blocking peptide (FIG. 1b). In human PBMCs, the expression of GHS-R on CD3⁺, CD3⁺ CD4⁺ and CD3⁺ CD8⁺ T cells was observed with no preferential expression pattern on these immune cell subsets. In highly purified human T cells, GHS-R expression significantly increases upon cellular activation (FIG. 1c), and in the presence of antibody-specific blocking peptide, this GHS-R labeling was almost completely ablated. Moreover, upon T-cell activation, there is also a marked up-regulation of GHS-R gene expression, as demonstrated by quantitative analysis of PCR products using Agilent gene chip technology and real time RT-PCR (FIG. 1d). The presence of GHS-R within lipid rafts and specific upregulation of GHS-R gene upon T cell activation shows a role for these receptors in T cell function. A similar staining pattern was observed for ghrelin receptors on activated T cells utilizing a second antibody recognizing 186-265 amino acid residues proximal to the C terminal region of human GHS-R (FIG. 8a).

Ligation of seven transmembrane GPCRs typically results in calcium mobilization from the intracellular stores by generation of inositol triphosphate (Kojima et al. (1999), Sherman-Baust et al. (2003)). Ghrelin has previously been shown to induce intracellular calcium release in GHS-R-transfected Chinese Hamster Ovary (CHO) cells (Kojima et al. (1999)). Here, using cultured human T cells, a significant and specific rise in intracellular [Ca²⁺] was demonstrated in response to both full-length ghrelin peptide (FIG. 1e) as well as ghrelin 1-18 fragment. This ghrelin-induced calcium flux was found to be GHS-R-specific as pretreatment with [D-Lys-3]-GHRP-6, a highly selective GHS-R antagonist, markedly attenuated the ghrelin-mediated intracellular calcium release from T cells (FIG. 1e). Interestingly, the intracellular calcium mobilization induced by ghrelin treatment is similar in magnitude to that observed in response to the positive control, stromal cell derived factor 1 (SDF-1), a potent T cell chemokine ligand that specifically binds and signals through the cell surface GPCR, CXCR4 (Sanchez-Madrid et al. EMBO J. 18: 501-511 (1999)). In addition to calcium mobilization, ligation of GPCRs is often accompanied by a dramatic remodeling of the actin cytoskeleton and cell surface molecules and leads to polarization and, in many cases, the directional migration of immune cells (Taub et al. (1993), Inui, A (1999)). Here, ghrelin induced a marked increase in broad membrane structures characteristic of lamellipodia with typical polarization of F-actin in a manner quite similar to the SDF-1-treated cells (FIG. 1f). Together, these data demonstrate the presence of functional GHS-R on the surface of human T cells and mononuclear cell subsets and support a biological role for ghrelin and GHS-R within the immune system.

### Example 3

### Ghrelin Receptor mRNA and Protein is Expressed in Human Monocytes

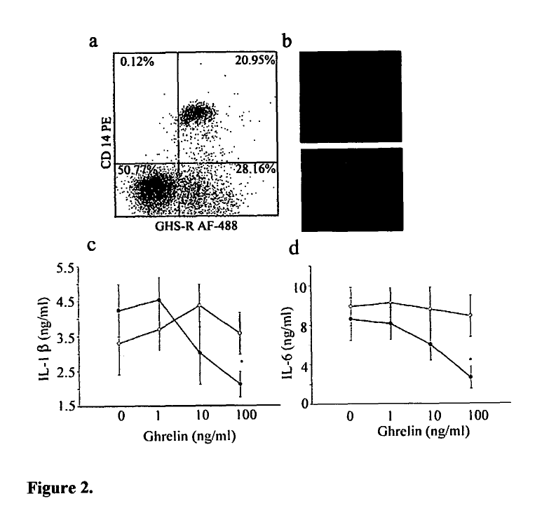

Among the mononuclear cells, monocytes constitute an important source of pro-inflammatory cytokines, prompting us to examine the GHS-R expression on monocytes. Flow cytometric analysis revealed approximately 21% CD14⁺ cells express GHS-R (FIG. 2a). Using immunofluorescence microscopy, diffuse GHS-R expression was detected on the cell surface of purified monocytes (2b upper), and control IgG demonstrated no specific labeling (FIG. 2b lower). Similarly, GHS-R expression was observed in immature and mature monocyte derived dendritic cells. Real time RT-PCR analysis also demonstrated the presence of GHS-R1a mRNA in monocytes with similar expression levels to primary human T cells.

### Example 4

### Ghrelin Selectively Inhibits Pro-inflammatory Cytokine Expression

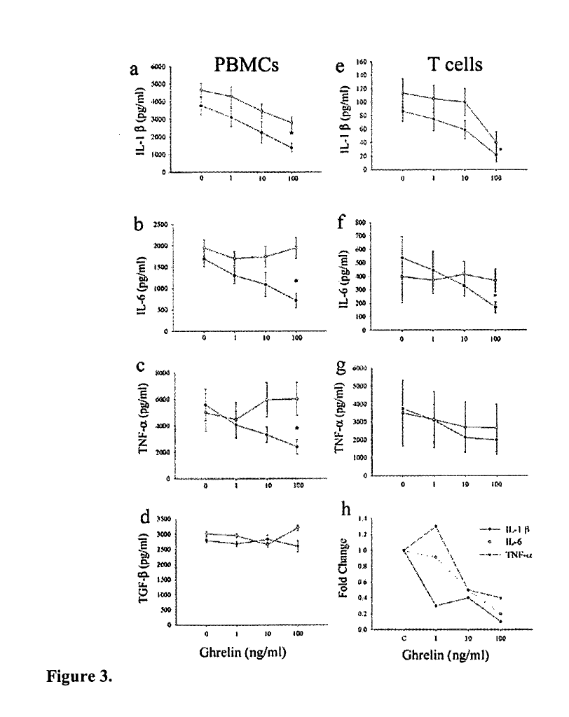

The classical pro-inflammatory cytokines, IL-1a, IL-1β, IL-6 and TNF-α are known to play a critical role in development of anorexia-cachexia syndrome (Inui et al. 1999. Cancer Res. 59: 4493-4501). The anorexia-cachexia syndrome is a complex multifactorial metabolic condition associated with altered protein, carbohydrate and fat metabolism resulting in anorexia, negative energy balance, weight loss and muscle wasting (Kotler et al. 2000. Ann. Internal Med. 133: 622-634). Considering the critical role played by pro-inflammatory cytokines in controlling metabolic activity, the ability of ghrelin to regulate the production of IL-1β, IL-6 and TNF-α by activated PBMCs and T cells was examined. Human PBMCs derived from healthy male subjects were stimulated with the polyclonal mitogen, phytohaemagglutinin (PHA), and incubated in the presence or absence of ghrelin and GHS-R antagonist for 24 h, after which supernatants were collected and examined for cytokine levels. Ghrelin treatment resulted in a significant inhibition of IL-1β, IL-6 and TNF-α production by PBMCs at ghrelin levels ranging from 1 to 100 ng/ml (FIG. 3a-c); however, ghrelin treatment failed to alter TGF-β production by these PBMCs at any concentration tested (FIG. 3d). This effect was found to be GHS-R-specific as the addition of GHS-R antagonist to these cultures attenuated this ghrelin-mediated inhibition and similar ghrelin effects on cytokine production were observed using concanavalin A (ConA)-stimulated PBMCs and LPS-treated monocytes. In addition, the primary human T cells stimulated with immobilized anti-CD3 antibody in presence of ghrelin for a 24 h time period demonstrated a significant dose-dependent inhibition of IL-1β and IL-6 (FIG. 3e-g). It should also be noted that this ghrelin-mediated inhibition was not due to any cytolytic effects of this hormone on T cells or PBMCs as measurement of lactate dehydrogenase (LDH) and cell counts using trypan blue exclusion failed to demonstrate any significant difference between control and hormone-treated cells. Using real time RT-PCR analysis, it was demonstrated that ghrelin significantly inhibits IL-1β, IL-6 and TNF-α mRNA expression in all the donors demonstrating a reduction in cytokine production (FIG. 3h). These results show ghrelin plays a role in the transcriptional regulation of inflammatory cytokine expression.

### Example 5

### Ghrelin Inhibits Leptin-Mediated Pro-inflammatory Cytokine Expression

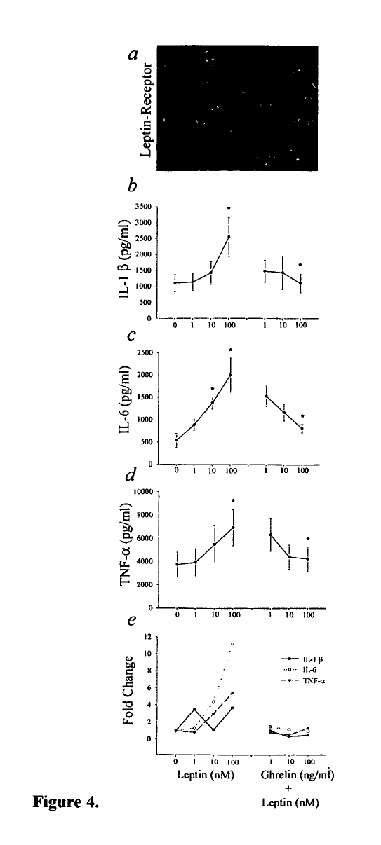

The mechanism of action by which leptin and ghrelin regulate inflammatory cytokine production was determined. The spatial localization of Ob-R protein on human T cells (FIG. 4a) was demonstrated, and it was also demonstrated that leptin directly induces a significant dose-dependent increase in IL-1β (FIG. 4b), IL-6 (FIG. 4c) and TNF-α (FIG. 4d) protein and mRNA expression by primary human T cells (FIG. 4e) and PBMCs. Upon concomitant addition of ghrelin to these cultures, a dose-dependent inhibition of leptin-induced cytokine protein and gene expression by T cells was observed in response to various concentrations of ghrelin (FIG. 4b-e). This shows that ghrelin and leptin, similar to their effects within the hypothalamus on food intake, exert mutually antagonistic effects on inflammatory cytokine expression within the immune system. Thus, the variations in circulating levels of leptin and ghrelin exert significant influence on the production of various cytokines by immune cell populations. Such reciprocal immunoregulatory effects are critical in maintaining immune cell homeostasis, therefore, preventing aberrant cytokine production, which results in or amplifies illness and pathology.

### Example 6

### Human T Cells Express and Actively Secrete Ghrelin

Ghrelin was thought to be exclusively produced by the stomach and subsequently secreted into the peripheral circulation (Kojima et al. 1999. Nature. 402: 656-660). However, it has been demonstrated that peripheral ghrelin levels gradually increase after gastrectomy, showing that additional cellular sources of ghrelin compensate for stomach-derived ghrelin (Dixit et al. 2003. Endocrinology. 144: 5595-5603).

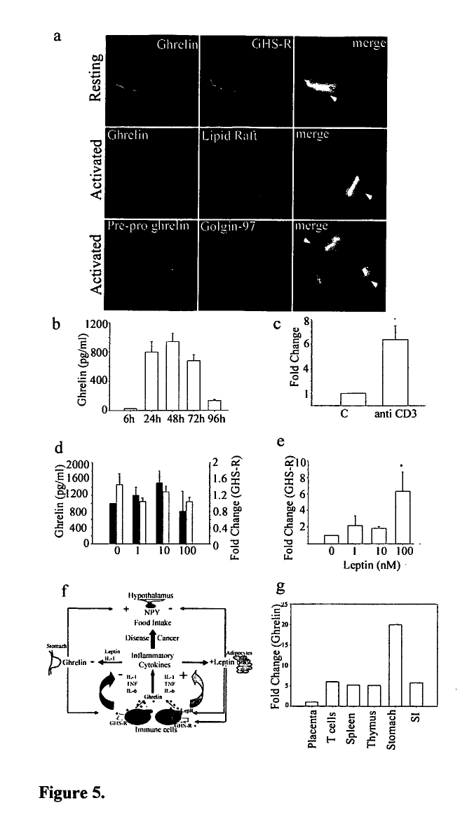

Lymphocytes are known to produce many well-characterized hormones like GH (Hosoda et al 2003. J. Biol. Chem. 278: 64-70), which exert a number of autocrine and paracrine effects on the immune system (Taub et al. 1994. J. Clin. Invest. 94: 293-300). Given the potent effect of ghrelin on cytokine expression, the possible presence of endogenously produced ghrelin by immune cells was hypothesized. The presence of immunoreactive ghrelin and GHS-R is demonstrated herein (FIG. 5a, upper) in resting human T cells with a broad distribution phenotype, with areas of co-localization showing ligand-receptor interaction and autocrine role for ghrelin in T cells. Upon TCR ligation, a distinct change in the spatial localization of the endogenous immunoreactive ghrelin was observed resulting in a polarized expression phenotype. Ghrelin appears to specifically associate within GM1⁺ lipid rafts (FIG. 5a, middle) showing that, upon activation, ghrelin is produced and specifically targeted towards lipid rafts and its own specific receptor. In further support of ghrelin synthesis by human T cells, it was found that the 117 amino acid pre-pro form of ghrelin is also co-expressed and co-localized within the Golgi apparatus (FIG. 5a, lower) where the pre-pro ghrelin is presumably cleaved and processed to its mature form prior to secretion. Both ghrelin and pre-pro-ghrelin staining in primary T cells is abolished upon addition of antibody-specific blocking peptide (FIG. 8d).

These findings are further supported by flow cytometric analysis of various T cell subsets for the mature ghrelin protein where the majority of T cells appeared to be ghrelin positive with no preferential expression in CD3⁺CD4⁺ or CD3⁺CD8⁺ T cell subsets (FIG. 5b). In addition to expression of intracellular ghrelin by T cells, TCR ligation of these cells results in substantial levels of ghrelin protein being secreted into the culture supernatant with levels peaking at 48 h and declining thereafter (FIG. 5b). Furthermore, T cell activation induced a greater than five fold increase in ghrelin mRNA expression as demonstrated by real time RT-PCR analysis (FIG. 5c).

Given the presence and production of ghrelin by T cells, ghrelin concentrations within the local microenvironment can reach significantly high levels without undergoing the classic dilution effect typically seen upon its release into the peripheral circulation from stomach. Thus, T cell-derived ghrelin can serve an important role in regulating cell function within an immune microenvironment or organ. Given the specific antagonistic effect of ghrelin on leptin-mediated inflammatory cytokine expression, the possible cross-regulatory effects of leptin on ghrelin and GHS-R expression in T cells was examined. Leptin failed to exert any significant effects on ghrelin protein production or gene expression within human T cell cultures (FIG. 5d). Furthermore, leptin treatment resulted in a significant increase in GHS-R mRNA expression by human T cells as measured by real time RT-PCR (FIG. 5e). Hence, the down-regulation of leptin-induced cytokine expression by ghrelin constitutes a reciprocal regulatory signaling pathway by which these hormones control each other's activities within the immune system (FIG. 5f). In addition, real time PCR analysis of a comparative ghrelin expression in human stomach and lymphoid organs revealed that stomach had an expression of 11 fold higher ghrelin than T cells, spleen and thymus (FIG. 5g). Lymphoid organs and small intestines expressed 5 fold higher ghrelin mRNA levels compared to placenta.

### Example 7

### Ghrelin Down-Regulates Inflammatory Cytokine Expression and Anorexia in Response to Endotoxin Challenge

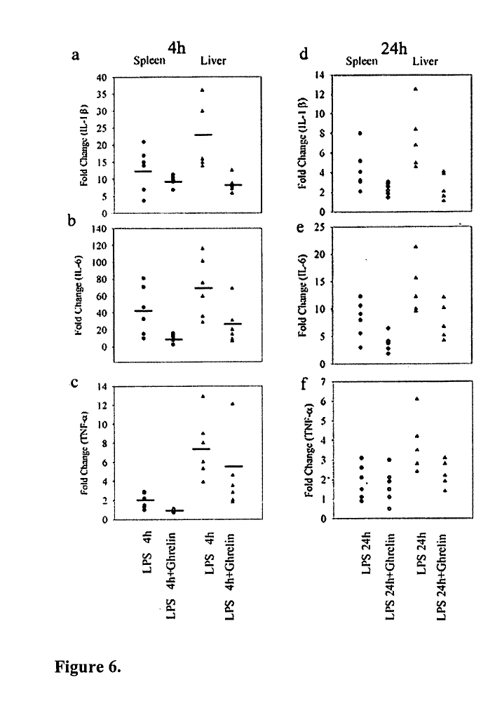

Bacterial lipopolysaccharide (LPS), the principal component in the pathogenesis of endotoxic shock, acts primarily on monocytes and evokes an acute phase response in vivo resulting in excessive production of IL-1β, IL-6 and TNF-α. The amplification of these proximal cytokines has a broad array of pro-inflammatory and anorexigenic effects (Kotler et al. 2000. Ann. Internal Med. 133: 622-634) contributing to pathogenesis of sepsis and multiple organ failure (Cohen et al. 2003. Nature 420: 885-891; Riedemann et al. 2003. J. Clin. Invest. 112: 460-467). In an effort to examine the ability of ghrelin to modulate inflammatory cytokine expression in vivo, mice were treated with ghrelin prior to and after LPS administration. As shown in FIG. 6, ghrelin exerted a potent anti-inflammatory effect on LPS-induced endotoxemia with inhibition of IL-1β, IL-6 and TNF-α expression in vivo. Real time PCR analysis of mRNA derived from the spleen and liver of these endotoxin-treated mice revealed a strong induction of these cytokine genes 4 h post LPS administration (FIG. 6a-c) with a significant diminishment in mRNA expression by 24 h (FIG. 6d-f). Mice treated with ghrelin and challenged with endotoxin demonstrated an attenuation of IL-1β and IL-6 mRNA expression in both spleen and liver after 4 and 24 h (FIG. 6a-f). Attenuation of TNF-α mRNA was observed in both spleen and liver at 4 h (FIG. 6c), TNF-α expression was also inhibited in liver 24 h post LPS and remained unchanged in spleen (FIG. 6f). Similar inhibition of pro-inflammatory cytokines was observed in lungs and mesenteric lymph nodes of ghrelin treated mice 4-24 h post LPS challenge.

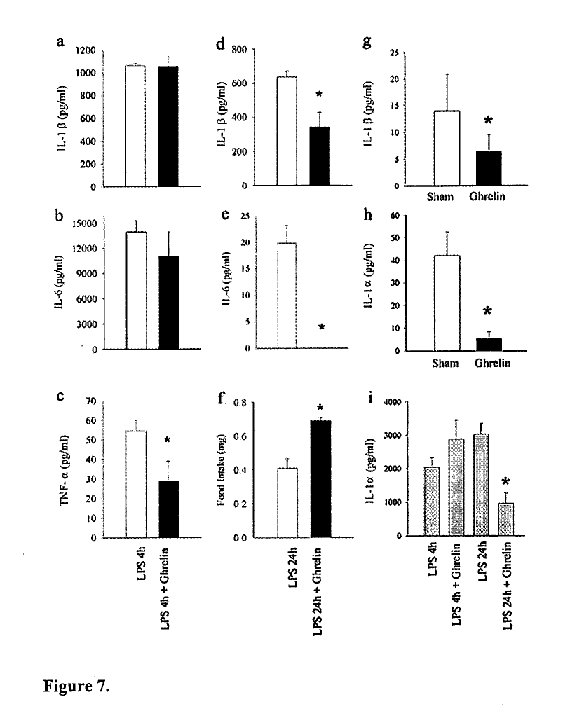

To measure circulating serum cytokine levels, mice were treated with LPS, and LPS followed by ghrelin treatment for either 4 or 24 hours. Analysis of the serum cytokine levels revealed a significant change in circulating TNF-α (FIG. 7c), but not in IL-1β (FIG. 7a) or IL-6 (FIG. 7b) levels at 4 h post ghrelin treatment; however, a significant inhibition of IL-1β and IL-6 was observed 24 h after LPS challenge (FIG. 7d,e). TNF-α levels were undetectable in the serum 24 h post LPS challenge. To examine the effects of ghrelin on endotoxin-induced anorexia, food intake was also assessed at 24 h post ghrelin and/or LPS administration. While the LPS challenged mice demonstrated a dramatic diminishment in food consumption compared to sham (80%), prior ghrelin treatment resulted in a significant attenuation of this LPS-induced anorexia (FIG. 7f). As expected ghrelin-treated control mice in the absence of LPS challenge, also demonstrated a significant increase in food intake (30%) compared to sham controls. Serum IL-1β and IL-1α levels were also significantly inhibited in these mice infused with ghrelin alone when compared to sham control mice (FIG. 7g,h), and serum ILα levels were inhibited 24 h post LPS and ghrelin treatment (FIG. 7h).

### Example 8

### Global Gene Expression Profile Associated with Ghrelin's Protective Effect in LPS Induced Murine Endotoxemia

Endotoxin shock in mice was induced by intraperitoneal (i.p.) injection with 10 μg of LPS (E. coli serotype 055:B5)). Animals also received a single i.p. injection of ghrelin (0.5 mg/kg) in PBS at 24 hours and 30 minutes prior to LPS administration. Mice were sacrificed 4 and 24 hours post LPS challenge, and visceral organs and serum were collected. RNA was extracted from spleens of sham and treated mice and utilized for gene array utilizing NIA murine 17K array. The hybridization spots on microarray filters were analyzed by using array pro software, and the average image intensity was then determined. The numerical intensities of each spot were normalized filterwide, and the relatively over- and underexpressed genes between various conditions were determined by a 1.5-fold mean ratio change.

Results: Gene expression was compared between the following conditions: 1) Sham vs Ghrelin; 2) LPS 4 h vs LPS 4 h+Ghrelin; and 3) LPS 24 h vs LPS24 h+Ghrelin.

Sham vs Ghrelin. There was a total of 71 upregulated known genes and 51 upregulated ESTs in the ghrelin gene array compared to the sham gene array. Of the anti-inflammatory target genes, the PACAP receptor and CD22 were both upregulated. There was a total of 134 downregulated known genes, and 94 downregulated ESTs. Anti-inflammatory target genes included lipoprotein lipase, fatty acid synthase, S-adenosyl homocysteine hydrolase, peripheral benzodiazepine receptor, TANK, serum glucocorticoid dinase (SGK), and lysophospholipase 1.

LPS 4 h vs LPS 4 h+Ghrelin. Gene expression was measured at a four hour time point in endotoxemic mice treated with ghrelin, and compared with endotoxemic mice not treated with ghrelin at the four hour time point. There was a total of 72 upregulated known genes, and 76 upregulated ESTs in the ghrelin array. Of the anti-inflammatory target genes, IGF-1, estrogen receptor 1 (alpha), and TIMP4 were upregulated. There was a total of 156 known genes that were downregulated, and 95 downregulated ESTs. Of the anti-inflammatory target genes, camodulin1, thioredoxin reductase, glutamate-cysteine ligase, carbonic anhydrase 2, squalene epoxidase, GAB1, and Trcp were down-regulated.

LPS 24 h vs LPS24 h+Ghrelin Gene expression was measured at a 24 hour time point in endotoxemic mice treated with ghrelin, and compared with endotoxemic mice not treated with ghrelin at the 24 hour time point. There was a total of 143 upregulated known genes and 118 upregulated ESTs. Of the anti-inflammatory target genes, Tom1 (target of Myb 1), thioredoxin reductase, glutamate-cysteine ligase (catalytic subunit) and glucocorticoid-induced gene 1 were detected. There was a total of 168 downregulated known genes, and 187 downregulated ESTs. Of the anti-inflammatory target genes, leukotriene A4 hydrolase, PECAM, LPS-induced TN factor, TANK, and Star were all detected.

Overall, in the endotoxemia model, inhibition of NFkB pathway was identified as one of the major mediators of ghrelin's protective effects. NFkB regulatory genes regulated by ghrelin were identified as TRCP, TOM1, AP2, GAB1 and TANK.

### Example 9

### Ghrelin Levels are Decreased in Subjects with Ulcerative Colitis and Crohn's Disease

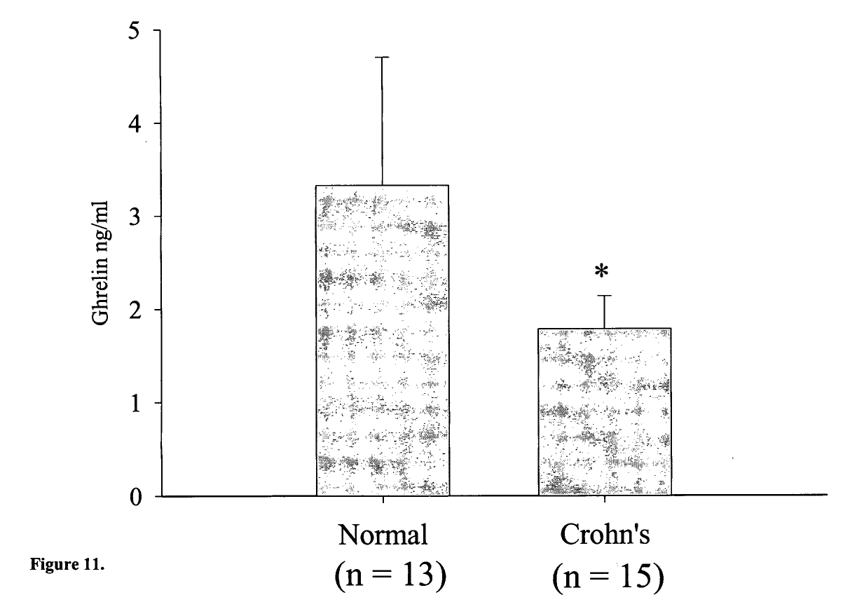

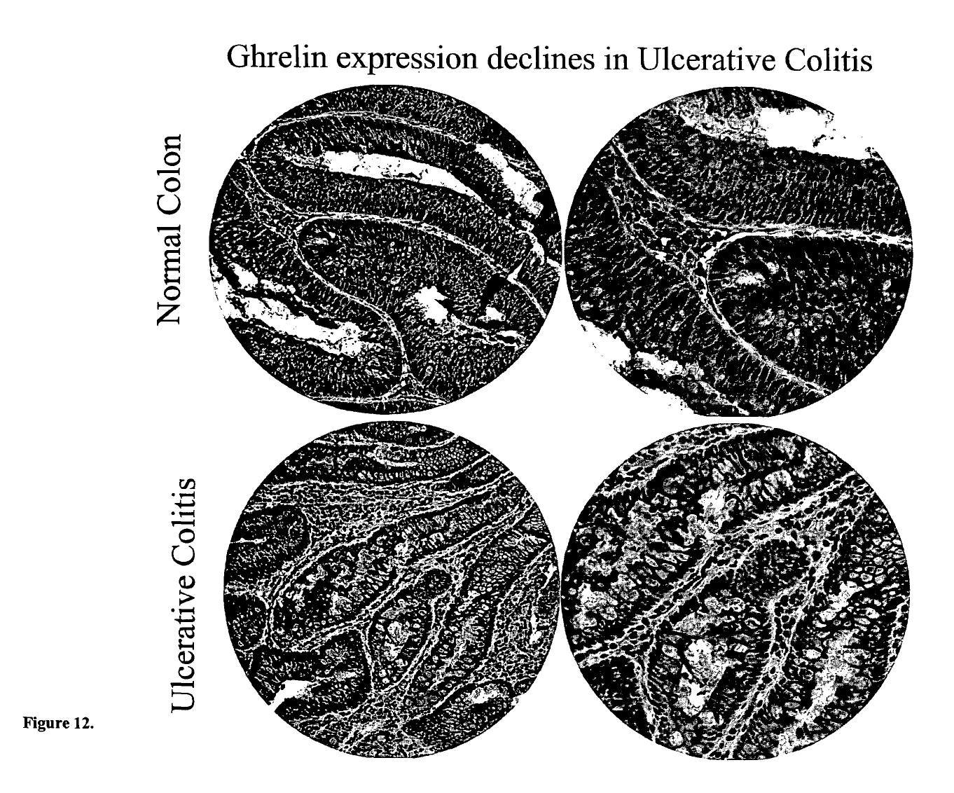

Serum ghrelin levels decline in patients with Crohn's disease (FIG. 11). In a study conducted with 15 subjects with Crohn's disease, and 13 subjects without, the level of ghrelin was found to be significantly lower in those subjects with Crohn's disease. Ghrelin expression is also significantly less in those subjects with ulcerative colitis (FIG. 12).

### REFERENCES

1. Kojima, M., Hosoda, H., Date, Y., Nakazato, M., Matsuo, H., and Kangawa K. 1999. Ghrelin is a growth-hormone-releasing acylated peptide from stomach. Nature. 402: 656-660.
2. Tschop, M., Smiley, D. L., and Heiman, M. L. 2000. Ghrelin induces adiposity in rodents. Nature. 407: 908-913.
3. Nakazato, M., Murakami, M., Date, Y., Kojima, M., Matsuo, H., Kangawa, K., and Matsukara, S. 2001. A role for ghrelin in the central regulation of feeding. Nature. 409: 194-198.
4. Cummings, D. E., Weigle, D. S., Frayo, R. S., Breen, P. A., Ma, M. K., Dellinger, E. P., and Purnell, J. Q. 2002. Human plasma ghrelin levels after diet-induced weight loss and gastric bypass surgery. New Engl. J. Med. 346: 1623-1630.
5. Inui, A. Ghrelin: An orexigenic and somatotrophic signal from the stomach. 2001. Nature Rev. Neurosci. 2: 551-560.
6. Howard, A. D. et al. A receptor in pituitary and hypothalamus that functions in growth hormone release. 1996. Science. 273: 974-977.
7. Palyha, O. C. et al Ligand activation of human orphan growth hormone (GH) secretagogue receptor (GHS-R) conserved from pufferfish to humans. 2000. Mol. Endocrinol. 14: 160-169.
8. Gnanapavan, S. et al. The tissue distribution of the mRNA of ghrelin and subtypes of its receptor, GHS-R, in humans. 2002. J. Clin. Endocrinol. Metab. 87: 2988-2991.
9. Poppi, L., Dixit, V. D., Baratta, M., Giustina, A., Tamanini, C., and Parvizi, M. 2002. Growth hormone secretagogue analogue hexarelin stimulates GH from peripheral lymphocytes. Exp. Clin. Endocrinol. Diabetes. 110: 343-347.
10. Dantzer, R. 2001. Cytokine induced sickness behaviour: Mechanisms and implications. Ann. NY. Acad. Sci. 933: 222-234.
11. Hart, B L. Biological basis of the behaviour of sick animals. 1988. Neurosci. Biobehav. Rev. 12: 123-137.
12. Kotler, D. P. Cachexia. 2000. Ann. Internal Med. 133: 622-634.
13. Ershler, W. B., and Keller, T. E. 2000. Age-associated increased interleukin-6 gene expression, late life diseases, and frailty. Annu. Rev. Med. 51: 245-270.
14. Bruunsgaard, H., Pedersen, M., and Pedersen, B. K. 2001. Ageing and pro-inflammatory cytokines. Curr. Opin. Hematol. 8: 131-136.
15. McCarty, M. F. 1999. Interleukin-6 as a central mediator of cardiovascular risk associated with chronic inflammation, smoking, diabetes, and visceral obesity: down regulation with essential fatty acids, ethanol, and pentoxifylline. Med. Hypotheses 52: 465-477.
16. Bochkov, V. N. Kadl, A., Huber, J., Gruber, F., Binder, B. R., and Leitinger, N. 2002. Protective role of phospholipid oxidation products in endotoxin-induced tissue damage. Nature. 419: 77-81.
17. Dixit, V. D., Sridaran, R., Edmonsond, M. A., Taub, D., and Thompson, W. E. 2003. Gonadotropin-releasing hormone attenuates pregnancy-associated thymic involution and modulates the expression of antiproliferative gene product prohibitin. Endocrinology. 144:1496-1505.
18. Nguyen, D. H., and Taub, D. 2002. Cholesterol is essential for macrophage inflammatory protein 1 beta binding and conformational integrity of CC chemokine receptor 5. Blood. 99: 4298-4306.
19. Sherman-Baust, C. A., Weeraratna, A. T., Rangel, L. B., Pizer, E. S., Cho, K. R., Schwartz, D. R., Shock, T., and Morin, P. J. 2003. Remodeling of the extracellular matrix through overexpression of collagen VI contributes to cisplatin resistance in ovarian cancer cells. Cancer Cell. 4: 377-386.
20. Nagasawa, T., Tachibana, K., and Kawabata, K. 1999. A CXC chemokine SDF-1/PBSF: a ligand for a HIV coreceptor, CXCR4. Adv. Immunol. 71: 211-228.
21. Sanchez-Madrid, F., and del Pozo, M. A. 1999. Leukocyte polarization in cell migration and immune interaction. EMBO J. 18: 501-511.
22. Taub, D D., Conlon, K., Lloyd, A. R., Oppenheim, J. J., and Kelvin, D. J. 1993. Preferential migration of activated CD4⁺ and CD8⁺ T cells in response to MIP-1 alpha and MIP-1 beta. Science. 260: 355-358.
23. Inui, A. Cancer anorexia-cachexia syndrome: are neuropeptides the key. 1999. Cancer Res. 59: 4493-4501.
24. Faggioni, R., Jones-Carson, J., Reed, D. A., Dinarello, C. A., Feingold, K. R., Grunfeld, C., and Fantuzzi, G. 2000. Leptin deficient (ob/ob) mice are protected from T cell-mediated hepatotoxicity: Role of tumor necrosis factor α and IL-18. Proc. Natl. Acad. Sci. USA. 97: 2367-2372.
25. Loffreda, S. et al. 1998. Leptin regulates pro-inflammatory immune responses. FASEB J. 12: 57-65.
26. Zarkesh-Esfahani, H., Pockley, G., Metcalfe, R. A., Bidlingmair, M., Wu, Z., Ajami, A., Weetman, A. P., Strasburger, C. J., and Ross, R. J. 2001. High dose leptin activates human leukocytes via receptor expression on monocytes. J. Immunol. 167: 4593-4599.
27. Lord, G. M., Matarese, G., Howard, J. K., Baker, R. J., Bloom, S. R., and Lechler R. I. 1998. Leptin modulates the T-cell immune response and reverses starvation-induced immunosuppression. Nature. 394: 897-901.
28. Hosoda, H., Kojima, M., Mizushima, T., Shimizu, S., and Kangawa K. 2003. Structural divergence of human ghrelin; identification of multiple ghrelin-derived molecules produced by post-translational processing. J. Biol. Chem. 278: 64-70.
29. Dixit, V. D., Mielenz, M., Taub, D., and Parvizi, N. 2003. Leptin induces growth hormone secretion from peripheral blood mononuclear cells via a protein kinase C and nitric oxide dependent mechanism. Endocrinology. 144: 5595-5603.
30. Taub, D. D., Tsarfaty, G., Lloyd, A. R., Durum, S. K., Longo, D. L., and Murphy, W. J. 1994. Growth hormone promotes human T cell adhesion and migration to both human and murine matrix proteins in vitro and directly promotes xenogeneic engraftment. J. Clin. Invest. 94: 293-300.
31. Asakawa, A. et al. 2001. Ghrelin is an appetite-stimulatory signal from stomach with structural resemblance to motilin. Gastroentrology 120: 337-345 (2001).

32. Cohen, J The immunopathogenesis of sepsis. 2003. Nature 420: 885-891.
33. Riedemann, N. C, Guo, R. F, and Ward, P. A. 2003. The enigma of sepsis. J. Clin. Invest. 112: 460-467.
34. Luheshi, G. N., Gardner, J. D., Rushforth, D. A., Loudon, A. S., and Rothwell, N. J. 1999. Leptin actions on food intake and body temperature are mediated by IL-1. Proc. Natl. Acad. Sci. USA. 96: 7047-7052.
35. Janik, J. E., Curti, B. D., Considine, R. V., Rager, H. C., Powers, G. C., Alvord, W. G., Smith, J. W 2^{nd}., Gause, B. L., and Kopp, W. C. 1997. Interleukin 1α increases serum leptin concentration in humans. J. Clin. Endocrinol. Metab. 82: 3084-3086.
36. Lambert, P. D. et al. 2001. Ciliary neurotrophic factor activates leptin-like pathways and reduces body fat, without cachexia or rebound weight gain, even in leptin-resistant obesity. Proc. Natl. Acad. Sci. USA. 98: 4652-4657.
37. Beretta, E., Dhillon, H., Kalra, P. S., and Kalra S P. 2002. Central LIF gene therapy suppresses food intake, body weight, serum leptin and insulin for extended periods. Peptides. 23: 975-984.
38. Wallenius, V., Wallenius, K., Ahren, B., Rudling, M., Carlsten, H., Dickson, S. L., Ohlsson, C., and Jansson, J. O. 2002. Interleukin-6-deficient mice develop mature-onset obesity. Nature Med. 8: 75-79.
39. Laviano, A., Russo, M., Freda, F., and Rossi-Fanelli, F. 2002. Neurochemical mechanisms for cancer anorexia. Nutrition. 18: 100-105.
40. Doehner, W. et al. Leptin, insulin sensitivity and growth hormone binding protein in chronic heart failure with and without cardiac cachexia. 2001. Eur. J. Endocrinol. 145: 727-735.
41. Nagaya, N. et al. 2001. Chronic administration of ghrelin improves left ventricular dysfunction and attenuates development of cardiac cachexia in rats with heart failure. Circulation. 104: 1430-1435.
42. Van den Berghe, G., Wouters, P., Weekers, F., Mohan, S., Baxter, R. C., Veldhuis, J. D., Bowers, C. Y., and Bouillon, R. 1999. Reactivation of pituitary hormone release and metabolic improvement by infusion of growth hormone-releasing peptide and thyrotropin-releasing hormone in patients with protracted critical illness. J. Clin. Endocrinol. Metab. 84: 1311-1323.
43. Sanna, V., Di Giacomo, A., La Cava, A., Lechler, R. I., Fontana, S., Zappacosta, S., and Matarese, G. 2003. Leptin surge precedes onset of autoimmune encephalomyelitis and correlates with development of pathogenic T cell responses. J. Clin. Invest. 111: 241-250.
44. Sun, Y., Ahmed, S., and Smith, R. G. 2003. Deletion of ghrelin impairs neither growth nor appetite. Mol. Cell. Biol. 23: 7973-7981.
45. Qian, S. et al. 2002. Neither agouti-related protein nor neuropeptide Y is critically required for the regulation of energy homeostasis in mice. Mol. Cell. Biol. 22: 5037-5035.
46. McCabe, J. B., and Berthiaume, L. G. 2001. N-terminal acylation confers localization to cholesterol, sphingolipid-enriched membranes but not to lipid rafts/caveolae. Mol. Biol. Cell. 12: 3601-3617.
47. Basa, N. R., Wang, L., Arteaga, J. R., tieber, D., Livingston, E. H., and Tache, Y. 2003. Bacterial lipopolysaccharide shifts fasted plasma ghrelin to postprandial levels in rats. Neurosci. Lett. 343: 25-28.
48. Hataya, Y., Akamizu, T., Hosoda, H., Kanamoto, N., Moriyama, K., Kangawa, K., Takaya, K., and Nakao, K. 2003. Alterations of plasma ghrelin levels in rats with lipopolysaccharide-induced wasting syndrome and effects of ghrelin treatment on the syndrome. Endocrinology. 144: 5365-5371.
49. Murray, C. D., Kamm, M. A., Bloom, S. R., and Emmanuel, A. V. 2003. Ghrelin for the gastroenterologist: history and potential. Gastroenterology. 125: 1492-1502.
50. O'Neill L A, Dinarello C A. The IL-1 receptor/toll-like receptor superfamily: crucial receptors for inflammation and host defense. Immunol Today. 2000 May; 21 (5):206-9
51. Cohen, J The immunopathogenesis of sepsis. 2003. Nature 420: 885-891.
52. Berg D J, Kuhn R, Rajewsky K, Muller W, Menon S, Davidson N, Grunig G, Rennick D. Interleukin-10 is a central regulator of the response to LPS in murine models of endotoxic shock and the Shwartzman reaction but not endotoxin tolerance. J Clin Invest. 1995 November; 96(5):2339-47.
53. Howard M, Muchamuel T, Andrade S, Menon S. Interleukin 10 protects mice from lethal endotoxemia. J Exp Med. 1993 Apr. 1; 177(4):1205-8.

### SEQUENCES

| SEQ ID NO: 1 | |
|---|---|
| Full length Ghrelin | |
| (Genbank accession number AB029434) | |
| MPSPGTVCSLLLLGMLWLDLAMAGSSFLSPEHQRVQQRKESKKPPAKLQPRALAGWLRPEDGGQAEGA | |
| EDELEVRFNAPFDVGIKLSGVQYQQHSQALGKFLQDILWEEAKEAPADK | |
| SEQ ID NO: 2 | |
| Full length Ghrelin nucleic acid | |
| 1 | gcaggcccac ctgtctgcaa cccagctgag gccatgccct ccccagggac cgtctgcagc |
| 61 | ctcctgctcc tcggcatgct ctggctggac ttggccatgg caggctccag cttcctgagc |
| 121 | cctgaacacc agagagtcca gcagagaaag gagtcgaaga agccaccagc caagctgcag |
| 181 | ccccgagctc tagcaggctg gctccgcccg gaagatggag gtcaagcaga aggggcagag |
| 241 | gatgaactgg aagtccggtt caacgccccc tttgatgttg gaatcaagct gtcaggggtt |
| 301 | cagtaccagc agcacagcca ggccctgggg aagtttcttc aggacatcct ctgggaagag |
| 361 | gccaaagagg ccccagccga caagtgatcg cccacaagcc ttactcacct ctctctaagt |
| 421 | ttagaagcgc tcatctggct tttcgcttgc ttctgcagca actcccacga ctgttgtaca |
| 481 | agctcaggag gcgaataaat gttcaaactg t |

SEQ ID NO: 3
Ghrelin (Human, 1-18)
peptide
Gly-Ser-Ser(n-Octanoyl)-Phe-Leu-Ser-Pro-Glu-His-Gln-Arg-Val-Gln-Gln-
Arg-Lys-Glu-Ser-NH₂
SEQ ID NO: 4
Ghrelin (Human, 1-14)
Gly-Ser-Ser(n-Octanoyl)-Phe-Leu-Ser-Pro-Glu-His-Gln-Arg-Val-Gln-Gln-
OH
SEQ ID NO: 5
Ghrelin (Human, Rat, 1-10)
Gly-Ser-Ser(n-Octanoyl)-Phe-Leu-Ser-Pro-Glu-His-Gln-NH₂
SEQ ID NO: 6
Ghrelin (Human, Rat, 1-5)
Gly-Ser-Ser(n-Octanoyl)-Phe-Leu-NH₂

What is claimed is:

1. A method of treating inflammation in a subject comprising administering to the subject an effective amount of one or more of the following:
a) ghrelin or a variant, derivative, or analog thereof;
b) SEQ ID NO: 1, or a fragment, variant derivative, or analog thereof;
c) SEQ ID NO: 2, or a fragment, variant, derivative, or analog thereof.

2-3. (canceled)

4. The method of claim 1, wherein the inflammation is associated with an infectious process, liver toxicity, or an inflammatory disease.

5-9. (canceled)

10. The method of claim 4, wherein said liver toxicity is associated with cancer therapy.

11-22. (canceled)

23. A method of treating loss of appetite in a subject by administering to the subject an effective amount of ghrelin or a variant, derivative, or analog thereof.

24. The method of claim 23, wherein the loss of appetite is caused by atherosclerosis, inflammation, aging, or psychological disorders.

25. The method of claim 24, wherein the psychological disorder is anorexia-cachexia syndrome.

26-31. (canceled)

32. A method of treating sepsis in a subject comprising administering to the subject an effective amount of ghrelin or a variant, derivative, or analog thereof.

33. The method of claim 32, wherein the sepsis is endotoxemia.

34. The method of claim 32, wherein 1-50 mg/kg body weight of ghrelin or a variant, derivative, or analog thereof is administered to the subject.

35. The method of claim 32, wherein 1-15 mg/kg body weight of ghrelin or a variant, derivative, or analog thereof is administered to the subject.

36. The method of claim 32, wherein about 5 mg/kg body weight of ghrelin is administered to the subject.

37. A method of inhibiting secretion of cytokines comprising administering an effective amount of ghrelin or a variant, derivative, or analog thereof.

38. The method of claim 37, wherein the cytokines are inhibited at the site of inflammation.

39. The method of claim 37, wherein the cytokine is selected from the group consisting of IL-1, IL-6, TNF-α, INF-γ, IL-12 and p40.

40. The method of claim 37, wherein the cytokine is expressed by cells selected from the group consisting of T-cells, B-cells, dendritic cells, and mononuclear cells.

41. The method of claim 4, wherein the infectious process is a viral infection, a bacterial infection, a parasitic infection, or a fungal infection.
